(19) United States
(12) Patent Application Publication
(10) Pub. No.: US 2026/0069190 A1
(43) Pub. Date: Mar. 12, 2026

(54) ELECTROCARDIOGRAM-BASED LEFT VENTRICULAR DYSFUNCTION AND EJECTION FRACTION MONITORING

(71) Applicant: Medtronic, Inc., Minneapolis, MN (US)

(72) Inventors: Rodolphe Katra, Blaine, MN (US); Niranjan Chakravarthy, Singapore (SG)

(21) Appl. No.: 19/106,020

(22) PCT Filed: Aug. 28, 2023
(86) PCT No.: PCT/US2023/072995

§ 371 Date: Feb. 24, 2025

Related U.S. Application Data
(60) Provisional application No. 63/373,865, filed on Aug. 30, 2022.

(51) Int. Cl. A61B 5/364; A61B 5/00; A61B 5/02; A61B 5/024; A61B 5/0245; A61B 5/11; A61B 5/352; A61B 5/353; A61B 5/355

(52) U.S. Cl. CPC A61B 5/364; A61B 5/02028; A61B 5/02405; A61B 5/0245; A61B 5/1118; A61B 5/1126; A61B 5/352; A61B 5/353; A61B 5/355; A61B 5/746; A61B 2562/0219

(57) ABSTRACT

A medical device system includes a medical device comprising one or more electrodes and configured to generate electrical cardiac data based on a cardiac signal sensed from a patient via the one or more electrodes, a memory configured to store a machine learning model and a plurality of sets of training data, and processing circuitry in communication with the memory. The processing circuitry is configured to apply the machine learning model to the electrical cardiac data to determine a value of a metric of left ventricular (LV) dysfunction. The machine learning model is trained based on the plurality of sets of training data. Each set of training data of the plurality of sets of training data includes a set of training electrical cardiac data and information indicating one or more values of the metric of LV dysfunction corresponding to the set of training electrical cardiac data.

This application claims the benefit of U.S. Provisional Patent Application No. 63/373,865, filed on Aug. 30, 2022, the entire content of which is incorporated herein by reference.

### TECHNICAL FIELD

This disclosure is directed to medical devices and, more particularly, to systems including medical devices and methods of operating such systems for monitoring patient conditions.

### BACKGROUND

Some types of medical devices may be used to monitor one or more physiological parameters of a patient, such as physiological parameters associated with cardiac function. Such medical devices may include, or may be part of a system that includes, sensors that detect signals associated with such physiological parameters; e.g., heart rate parameters. Values determined based on such signals may be used to assist in detecting changes in medical conditions, in evaluating the efficacy of a therapy, or in generally evaluating patient health.

Medical devices that monitor physiological parameters related to a medical condition of a patient may evaluate values associated with the physiological parameters, such as to determine whether the values exceed a threshold or have changed over time. Values that exceed a threshold or that have changed may indicate a change, e.g., worsening, of a patient's medical condition, or that a therapy being administered to the patient is not effectively managing the patient's medical condition.

### SUMMARY

In general, this disclosure is directed to techniques for determining a metric of left ventricular (LV) dysfunction, such as LV ejection fraction (LVEF), based on an electrocardiogram (ECG) or an electrogram (EGM) sensed by a device, such as an insertable cardiac monitor (ICM), or another implantable medical device (IMD). LV dysfunction may be related to heart failure (HF). In some examples, processing circuitry of a system that implements the techniques of this disclosure may further determine HF status, e.g., determine or likelihood of future worsening HF, based on the metric of LV dysfunction.

According to the techniques of this disclosure, an insertable cardiac monitor (ICM) or other implantable medical device (IMD) may collect a cardiac signal that indicates electrical cardiac data such as ECG data or EGM data. Cardiac signals may include information that is relevant to monitoring a variety of patient conditions. For example, a cardiac signal may include fiducials that indicate events in the cardiac cycle. For example, a P-wave indicates an atrial depolarization, an R-wave indicates a ventricular depolarization, and a T-wave indicates a ventricular repolarization. The system may analyze the cardiac signal to identify these fiducials, or monitor one or more patient conditions based on identified fiducials and/or other information. For example, the system may classify the cardiac signal as indicating one or more patient conditions such as atrial fibrillation (AF).

The techniques of this disclosure may be implemented by systems including one or more IMDs that can autonomously and continuously collect biometric data and store biometric data while the one or more IMDs are implanted in a patient over months or years and perform numerous operations per second on the data to enable the systems herein to analyze the biometric data. Using techniques of this disclosure with an IMD may be advantageous when a physician cannot be continuously present with the patient to evaluate the physiological parameters and/or where performing the operations on the data described herein could not practically be performed in the mind of a physician.

In some examples, the techniques and systems of this disclosure may use one or more models, e.g., probability and/or machine learning models, to more accurately analyze biometric data to identify metric values, monitor a patient condition, or predict a future status of a patient condition. In some examples, the model is trained with a set of training instances, where one or more of the training instances comprise data that indicate relationships between biometric data and likelihood values. Because the model is configured based on potentially thousands or millions of training instances, the model may reduce the amount of error in recurrence prediction performed by the system. Reducing errors using the techniques of this disclosure may provide one or more technical and clinical advantages, such as increasing the likelihood that the system will accurately provide information based analyzing the biometric data.

A machine learning model may provide a more accurate output when the model is trained using a larger set of training data as compared with when the model is trained using a smaller set of training data. This is because larger training datasets provide a greater number of training instances than smaller training datasets provide, meaning that models trained using larger training datasets generate outputs based on a greater amount of knowledge as compared with models trained using smaller training datasets. When a machine learning model is trained using supervised learning, the training data is labelled so that the model learns associations between data features and labels. This means that an accuracy of a machine learning model trained using supervised learning may be limited when an amount of available labeled training data is limited.

In some examples, a health monitoring system may apply a machine learning model to electrical cardiac data generated by a medical device based on a cardiac signal sensed from a patient by the medical device to determine a value of a metric of LV dysfunction. When the health monitoring system applies the machine learning model to the electrical cardiac data, the machine learning model may process the electrical cardiac data to determine the value of a metric of LV dysfunction. The machine learning model may, in some examples, output a determined value of the metric of LV dysfunction. In some examples, the machine learning model may output a confidence that the metric of LV dysfunction is lower than a threshold metric of LV dysfunction and/or a confidence that the metric of LV dysfunction is not lower than a threshold metric of LV dysfunction.

One or more characteristics of a set of electrical cardiac data may indicate a value of a metric of LV dysfunction corresponding to the set of electrical cardiac data. For example, electrical cardiac data may indicate a heart rate, a heart rate variability, one or more arrhythmias, characteristics corresponding to R-Waves, P-waves, T-waves or other cardiac events, or any combination thereof. By applying the machine learning model to the electrical cardiac data that includes these characteristics, the health monitoring system may be configured to cause the machine learning model to output a value of a metric of LV dysfunction. It may be beneficial to use a machine learning model to generate a value of a metric of LV dysfunction based on electrical cardiac data so that the health monitoring system may determine the metric of LV dysfunction based on electrical cardiac data collected by a medical device.

In some examples, the machine learning model applied by the health monitoring system may be trained using a plurality of sets of training data. Each set of training data of the plurality of sets of training data may include a set of training electrical cardiac data and information indicating one or more values of the metric of LV dysfunction corresponding to the set of training electrical cardiac data. That is, each value of the one or more values of the metric of LV dysfunction indicated by a set of training data may be associated with a time relative to the set of training electrical cardiac data. This means that a set of training data may be labeled with one or more values of the metric of LV dysfunction at times relative to characteristics of the training electrical cardiac data. When the machine learning model is trained, the model may learn to recognize patterns corresponding to characteristics of the electrical cardiac data and the metric of LV dysfunction.

The techniques of this disclosure may provide one or more advantages. For example, by using a machine learning model trained with sets of training data that include both electrical cardiac data and one or more values of the metric of LV dysfunction corresponding to the electrical cardiac data to determine values of the metric of LV dysfunction, the system may more accurately determine values of the metric of LV dysfunction as compared with systems that do not use a machine learning model trained with electrical cardiac data labeled with values of the metric of LV dysfunction. The machine learning model may also be assessed on a patient-by-patient basis. For example, the system may determine whether the machine learning model performs adequately in determining values of the metric of LV dysfunction based on electrical cardiac data collected from a particular patient and update the model if performance is not adequate. Consequently, the machine learning model may perform better for individual patients as compared with systems that do not update a machine learning model based on performance on a patient-by-patient basis.

In one example, a medical device system includes a medical device comprising one or more electrodes and configured to generate electrical cardiac data based on a cardiac signal sensed from a patient via the one or more electrodes, a memory configured to store a machine learning model and a plurality of sets of training data, and processing circuitry in communication with the memory. The processing circuitry is configured to apply the machine learning model to the electrical cardiac data to determine a value of a metric of left ventricular (LV) dysfunction, wherein the machine learning model is trained based on the plurality of sets of training data, wherein each set of training data of the plurality of sets of training data includes a set of training electrical cardiac data and information indicating one or more values of the metric of LV dysfunction corresponding to the set of training electrical cardiac data. The processing circuitry is also configured to output the determined value of the metric of LV dysfunction to a computing device.

In another example, a method of operating a medical device system comprising a medical device comprising one or more electrodes and configured to generate electrical cardiac data based on a cardiac signal sensed from a patient via the one or more electrodes includes applying, by processing circuitry of the medical device system, a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data. The method also includes outputting, by the processing circuitry, the determined value of the metric of LV dysfunction to a computing device.

In another example, a non-transitory computer-readable storage medium includes program instructions that, when executed by processing circuitry of a medical device system comprising one or more electrodes and configured to generate electrical cardiac data based on a cardiac signal sensed from a patient via the one or more electrodes, cause the processing circuitry to apply a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data. The program instructions also cause the processing circuitry to output the determined value of the metric of LV dysfunction to a computing device.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is a block diagram illustrating an example medical device system including an implantable medical device (IMD) in conjunction with a patient, in accordance with one or more techniques of this disclosure.

FIG. 2A is a perspective drawing illustrating an IMD, which may be an example configuration of the IMD of FIG. 1 as an insertable cardiac monitor (ICM), in accordance with one or more techniques of this disclosure.

FIG. 2B is a perspective drawing illustrating another IMD, which may be another example configuration of the IMD from FIG. 1 as an ICM, in accordance with one or more techniques of this disclosure.

FIG. 3 is a block diagram illustrating an example configuration of the IMD of FIG. 1, in accordance with one or more techniques of this disclosure.

FIG. 4 is a block diagram illustrating an example configuration of a computing device, which may correspond to either (or both operating in coordination) of the computing devices of FIG. 1, in accordance with one or more techniques of this disclosure.

FIG. 5 is a block diagram illustrating an operating perspective of the health monitoring system (HMS) of FIG. 1, in accordance with one or more techniques of this disclosure.

FIG. 6 is a conceptual diagram illustrating an example machine learning model configured to output information corresponding to electrical cardiac data, in accordance with one or more techniques of this disclosure.

FIG. 7 is a block diagram illustrating an example of a machine learning model being trained using supervised and/or reinforcement learning, in accordance with one or more techniques of this disclosure.

FIG. 8 is a flow diagram illustrating an example method for determining whether to use a machine learning model to process electrical cardiac data collected from a patient, in accordance with one or more techniques of this disclosure.

FIG. 9 is a flow diagram illustrating an example method for applying a machine learning model to determine a value of a metric of left ventricle (LV) dysfunction, in accordance with one or more techniques of this disclosure.

### DETAILED DESCRIPTION

A device comprising one or more physiological sensors includes at least electrodes to sense at least one channel of electrical cardiac data such as electrocardiogram (ECG) data and/or electrogram (EGM) data. Processing circuitry of the device, or of another device in communication with the sensing device, determines a metric of left ventricular (LV) dysfunction, such as LV ejection fraction (LVEF), based on the electrical cardiac data. In some examples, the device may be an implantable medical device (IMD). The IMD may include a housing, configured for subcutaneous implantation, on which the one or more electrodes are positioned, such as an insertable cardiac monitor (ICM). In some examples, the IMD may be a leadless IMD. In other examples, the device may be one or more other implanted or external devices. Examples of the one or more other implanted or external devices may include an implantable, multi-channel cardiac pacemaker, implantable cardioverter-defibrillator (ICD), implantable pulse generator (IPG), leadless (e.g., intracardiac) pacemaker, extravascular pacemaker and/or ICD, or other IMD or combination of such IMDs, an external monitor, a drug pump, or a smartwatch or other consumer device with physiological monitoring capabilities.

In any such examples, the processing circuitry may transmit the LV dysfunction metric values of the patient to a remote computer, receive instructions from the remote computer for a medical intervention based on a heart failure status of the patient, and transmit the instructions for the medical intervention to a user interface. Such instructions for a medical intervention may include at least one of a change in a drug selection, a change in a drug dosage, instructions to schedule a visit with a clinician, or instructions for the patient to seek medical attention. In this manner, a patient's diagnoses and/or treatment for a condition may be modified as needed in between clinic visits, which may help avoid adverse medical events such as recurrent symptoms, acute heart failure (HF) decompensation, or hospitalization.

In some examples, the techniques described herein may enable identification of changes in a status of a patient before the changes lead to symptoms, and/or the progression or the patient's condition or development of one or more additional medical conditions. Thus, the techniques described herein may help enable determination of possibility that the patient will experience an adverse medical event, which may help clinicians prescribe personalized treatment to help avert hospitalizations, improve clinical outcomes, and/or reduce the economic burden on the health care system.

This disclosure describes various systems, devices, and techniques for analyzing biometric data collected by a medical device to generate information relating to a health of a patient. In some examples, it may be possible to identify one or more patient conditions, such as HF, a cardiac arrhythmia, or predict a future occurrence of one or more patient conditions based on analyzing electrical cardiac data such as an ECG or an EGM. A system may use a machine learning model to analyze cardiac data in order to generate information relating to a patient condition such as one or more values of a metric of LV dysfunction. In some cases, the system may train the machine learning model using a large set of training data that includes one or both of unlabeled training datasets and labeled training datasets.

In machine learning, “labeled” training data refers to training data that has information attached identifying one or more aspects of the data. For example, electrical cardiac data such as ECG data and EGM data may include labels identifying P-waves, R-waves, and T-waves. In another example, electrical cardiac data may include labels indicating parameters such as heart rate, heart rate variability, or characteristics such as arrythmias. In another example, electrical cardiac data may be labeled with one or more values of a metric of LV dysfunction, such as LVEF. A label including a value of a metric of LV dysfunction may indicate a time of the value of the metric of LV relative to one or more times of the electrical cardiac data. Other example labels may include patient information such as age, sex, preexisting conditions, or any combination thereof. “Unlabeled” training data refers to training data that does not include information identifying aspects of the data. An unlabeled electrical cardiac training dataset may include electrical cardiac data including features such as P-waves, R-waves, and T-waves without any labels that identify such features or any other characteristics of the dataset.

A system may be configured to train the machine learning model based on a plurality of sets of training data. Each set of training data may include a set of training electrical cardiac data and information indicating one or more values of the metric of LV dysfunction corresponding to the set of training electrical cardiac data. That is, the set of training electrical cardiac data may include one or more labels indicating values of the metric of LV dysfunction. The set of training electrical cardiac data may include one or more labels indicating characteristics of the electrical cardiac data, but this is not required. The system may train the machine learning model based on a plurality of sets of training data so that the machine learning model learns patterns corresponding to the electrical cardiac data and the metric of LV dysfunction.

LVEF is a clinically actionable cardiac function metric that may be measured in clinical settings using echocardiogram, cardiac magnetic resonance imaging (MRI), or other modalities. In some examples, it may be beneficial to derive a metric of LV dysfunction such as LVEF based on electrical cardiac data such as ECG data or EGM data. This is because an IMD may record electrical cardiac data from a patient continuously over an extended period of time, and deriving a metric of LV dysfunction such as LVEF based on electrical cardiac data may allow a system to track the metric of LV dysfunction over the extended period of time without a need for the patient to make frequent visits to a clinic for echocardiogram and/or cardiac MRI measurements.

Using electrical cardiac data to monitor a metric of LV dysfunction may be beneficial because electrical cardiac data such as ECG data and EGM data has diagnostic relevance for indicating metrics of LV dysfunction such as LVEF. Using electrical cardiac data to monitor a metric of LV dysfunction may allow a system to achieve continuous monitoring of the metric of LV dysfunction over a longer period of time as compared with systems that use methods such as an echocardiogram and/or MRI. Using electrical cardiac data to monitor a metric of LV dysfunction may allow a system to monitor a metric of LV dysfunction more accurately in real-time and/or via cloud-based artificial intelligence (AI) over an extended period of time as compared with systems that use echocardiogram and/or MRI to measure the metric of LV dysfunction, because electrical cardiac data may be easier to record over an extended period of time by an IMD as compared with echocardiogram data and/or MRI data.

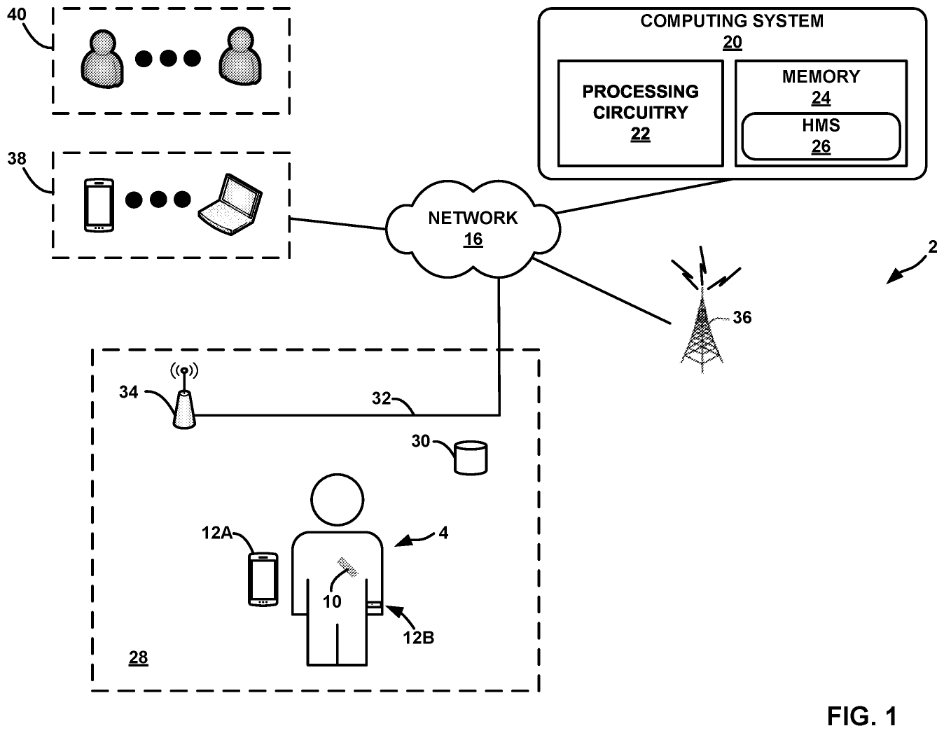

FIG. 1 is a block diagram illustrating an example medical device system 2 including an IMD 10 in conjunction with a patient 4, in accordance with one or more techniques of this disclosure. IMD 10 is configured for continuous, long-term monitoring of the heart of patient 4. For example, IMD 10 is configured to sense a cardiac signal indicating electrical cardiac data such as ECG data, EGM data, or other electrical cardiac data, identify fiducials in the cardiac signal, identify one or more parameter values (e.g., a value of a metric of LV dysfunction) based on the cardiac signal, detect a patient condition or arrythmia (e.g., HF, ventricular fibrillation (VF), atrial fibrillation (AF), atrioventricular (AV) block) based on the cardiac signal, store the cardiac signal and information corresponding to the cardiac signal to a memory of IMD 10, or any combination thereof. In some examples, IMD 10 is configured to automatically sense the cardiac signal continuously or intermittently according to a predetermined schedule. In some examples, IMD 10 is configured to sense the cardiac signal based on user input. For example, IMD 10 may sense the patient signal based on a user input indicating a symptom of a patient condition.

As examples, IMD 10 may be a pacemaker or implantable cardioverter-defibrillator, which may be coupled to intravascular or extravascular leads, or a pacemaker with a housing configured for implantation within the heart, which may be leadless. Some IMDs do not provide therapy, such as implantable patient monitors. IMD 10 may be such an IMD, e.g., the Reveal LINQ™ or LINQ II™ Insertable Cardiac Monitor (ICM), available from Medtronic, Inc., of Minneapolis, Minnesota, which may be inserted subcutaneously. Such IMDs may facilitate relatively longer-term and continuous monitoring of patients during normal daily activities, and may periodically transmit collected data to a remote patient monitoring system, such as the Medtronic CareLink™ Network.

IMD 10 may determine values of patient parameters or metrics, e.g., based on physiological signals sensed by the IMD or responsive therapies delivered by the IMD. The patient parameters may include, as examples, fluid level, heart rate, respiration rate, patient activity, temperature, heart sounds, oxygenation, and R-wave morphological characteristics. Other example patient metrics include coughing, speech, posture, tissue perfusion, hematocrit, thoracic impedance, subcutaneous impedance, intracardiac impedance, heart rate variability (HRV), weight, blood pressure, sleep apnea burden (which may be derived from respiration rate), ischemia burden, sleep duration, sleep quality, pre-ventricular contraction (PVC) burden, the occurrence, a metric of LV dysfunction such as LVEF, and frequency or duration cardiac arrhythmias or other events, such as HF, VF, AF or AV block, and sensed cardiac intervals (e.g., Q-T intervals). Another example patient metric is the ventricular rate during AF. The concentration or levels of various substances, such as blood glucose, hematocrit, troponin and/or brain natriuretic peptide (BNP) levels, within the patient may also be used as one or more patient metrics.

Medical device system 2 may include one or more sensors (e.g., for sensing an activity state of patient 4 and/or cardiac function of patient 4). The one or more sensors collectively may detect at least one first signal and at least one second signal that enable a processing circuitry of medical device system 2 to determine whether an activity state of patient 4 satisfies at least one inactivity criterion and determine values of at least one LV dysfunction metric, such as LVEF, based on such signals. Although such processing circuitry may be contained within IMD 10 and/or within another medical device of medical device system 2, e.g., external device 12, the processing circuitry may be described herein as being a component of IMD 10 for the sake of clarity.

In some examples, the one or more sensors may include one or more accelerometers or other sensors configured to detect the at least one signal indicative of one or more aspects of an activity state of patient 4, such as activity level, posture, and/or respiration rate. In some examples, the one or more sensors may include a plurality of electrodes, which may be positioned on the housing of IMD 10. The plurality of electrodes may be configured to detect an electrical cardiac signals such as an ECG and/or an EGM.

In addition to IMD 10, medical device system 2 includes one or more patient computing devices, e.g., patient computing devices 12A and 12B (collectively, “patient computing devices 12”). Patient computing device(s) 12 are configured for wireless communication with IMD 10. Computing device(s) 12 may retrieve parameter data, ECG data and/or EGM data, LV dysfunction metric data and other data from IMD 10. In some examples, computing device(s) 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing device(s) 12 may communicate with IMD 10 and each other according to the Bluetooth® or Bluetooth® Low Energy (BLE) protocols, as examples. In some examples, only one of computing device(s) 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store parameter data based on such signals. Computing device 12B may be incorporated into the apparel of patient 4, such as within clothing, shoes, eyeglasses, a watch or wristband, a hat, etc. In some examples, computing device 12B is a smartwatch or other accessory or peripheral for a smartphone computing device 12A.

One or more of computing device(s) 12 may be configured to communicate with a variety of other devices or systems via a network 16. For example, one or more of computing device(s) 12 may be configured to communicate with one or more computing systems, e.g., computing system 20, via network 16. Computing system 20 may be managed by a manufacturer of IMD 10 to, for example, provide cloud storage and analysis of collected data, maintenance and software services, or other networked functionality for their respective devices and users thereof. Computing system 20 may comprise, or may be implemented by, the Medtronic CareLink™ Network, in some examples.

As illustrated in FIG. 1, computing system 20 may include processing circuitry 22 and memory 24. Processing circuitry 22 may include fixed function circuitry and/or programmable processing circuitry. Processing circuitry 22 may include any one or more of a microprocessor, a controller, a digital signal processor (DSP), graphics processing unit (GPU), tensor processing unit (TPU), an application specific integrated circuit (ASIC), a field-programmable gate array (FPGA), or equivalent discrete or analog logic circuitry. In some examples, processing circuitry 22 may include multiple components, such as any combination of one or more microprocessors, one or more controllers, one or more DSPs, GPUs, TPUs, one or more ASICs, or one or more FPGAs, as well as other discrete or integrated logic circuitry, which may be physically located in one or more devices in one or more physical locations. Computing system 20 may be configured as a cloud computing system.

Processing circuitry 22 may be capable of processing instructions stored in memory 24. In some examples, memory 24 includes a computer-readable medium that includes instructions that, when executed by processing circuitry 22, cause computing system 20 and processing circuitry 22 to perform various functions attributed to them herein. In the example illustrated by FIG. 1, computing system 20 implements a health monitoring system (HMS) 26. As will be described in greater detail below, HMS 26 may generate information corresponding to one or more patient conditions of patient 4 based on physiological data collected by IMD 10. Memory 24 may include any volatile, non-volatile, magnetic, optical, or electrical media, such as a random-access memory (RAM), read-only memory (ROM), non-volatile RAM (NVRAM), electrically erasable programmable ROM (EEPROM), ferroelectric RAM (FRAM), dynamic random-access memory (DRAM), flash memory, or any other digital media.

Computing device(s) 12 may transmit data, including data retrieved from IMD 10, to computing system 20 via network 16. The data may include sensed data, e.g., values of physiological parameters measured by IMD 10 and, in some cases one or more of computing device(s) 12, and other physiological signals or data recorded by IMD 10 and/or computing device(s) 12. The data transmitted from computing device(s) 12 to computing system 20 may include electrical cardiac data such as ECG data and/or EGM data.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to generate diagnostic information of patient 4, such as information indicating one or more values of a metric of LV dysfunction. In some examples, IMD 10 may be configured to transmit such data to wireless access point 34 and/or computing device(s) 12. Wireless access points 34 and/or computing device(s) 12 may then communicate the retrieved data to computing systems 20 via network 16.

In some cases, computing system 20 may be configured to provide a secure storage site for data that has been collected from IMD 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians 40, via clinician computing devices 38. One or more aspects of the example system described with reference to FIG. 1 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a status of a medical condition. For example, computing system 20 may transmit data indicating one or more sets of electric cardiac data and/or one or more values of a metric of LV dysfunction (e.g., determined by computing system 20, computing device 12, or other devices described herein) to clinicians 40 via clinician computing devices 38.

As will be described herein, processing circuitry of one or more devices of medical device system 2 (e.g., of computing device 12 and/or computing system 20 implementing HMS 26) may be configured to determine information corresponding to electrical cardiac data (e.g., ECG data and/or EGM data) generated by IMD 10. In some examples, HMS 26 may include a machine learning model that is configured to generate an output based on receiving electrical cardiac data generated by IMD 10 as an input. The machine learning model may, in some examples, generate an output that includes information corresponding to a health of patient 4. For example, the machine learning model may output one or more values of a metric of LV dysfunction corresponding to electrical cardiac data generated by IMD 10. In some cases, the machine learning model may output information indicating that patient 4 is likely to experience a level of the metric of LV dysfunction in the future. In some examples, the machine learning model may be stored by a memory of IMD 10, but this is not required. The machine learning model may be stored by a device separate from IMD 10.

In some examples, computing system 20 may be configured to determine a long-term trend of the metric of LV dysfunction on a patient-specific basis. For example, computing system 20 may determine a historical estimate of the metric of LV dysfunction based on values of the metric of LV dysfunction determined based on electrical cardiac data collected by IMD 10 and actual values of the metric of LV dysfunction measured via echocardiogram and/or a cardiac MRI. For example, computing system 20 may be configured to determine a long-term trend of the metric of LV dysfunction based on values of the LV dysfunction determined based on electrical cardiac data collected by IMD 10 and actual values of the metric of LV dysfunction on a patient-specific basis using data corresponding to a given patient.

In the example of FIG. 1, environment 28 includes one or more Internet of Things (IoT) devices, such as IoT device 30. IoT device 30 may include, as examples, so called “smart” speakers, cameras, televisions, lights, locks, thermostats, appliances, actuators, controllers, or any other smart home (or building) devices. In the example of FIG. 1, IoT device 30 is a smart speaker and/or controller, which may include a display. In some examples, IoT device 30 includes cameras or other sensors may activate those sensors to collect data regarding patient 4, e.g., for evaluation of the condition of patient 4.

Computing device(s) 12 may be configured to wirelessly communicate with IoT device 30 to cause IoT device 30 to take the actions described herein. In some examples, HMS 26 communicates with IoT device 30 via network 16 to cause IoT device 30 to take the actions described herein. In some examples, IMD 10 is configured to communicate wirelessly with IoT device 30, e.g., to communicate data to computing system 20 via network 16. In some examples, IoT device 30 may be configured to provide some or all of the functionality ascribed to computing device(s) 12 herein.

Environment 28 includes computing facilities, e.g., a local network 32, by which computing device(s) 12, IoT device 30, and other devices within environment 28 may communicate via network 16, e.g., with HMS 26. For example, environment 28 may be configured with wireless technology, such as the Institute of Electrical and Electronics Engineers (IEEE) 802.11 wireless networks, IEEE 802.15 ZigBee networks, an ultra-wideband protocol, near-field communication, or the like. Environment 28 may include one or more wireless access points, e.g., wireless access point 34 that provides support for wireless communications throughout environment 28. Additionally, or alternatively, e.g., when local network is unavailable, computing device(s) 12, IoT devices 30, and other devices within environment 28 may be configured to communicate with network 16, e.g., with HMS 26, via a cellular base station 36 and a cellular network.

In some examples, computing device(s) 12 and/or computing system 20 may implement one or more algorithms to determine information corresponding to patient 4 based on data received from IMD 10. In some examples, computing device(s) 12 and/or computing system 20 may have greater processing capacity than IMD 10, enabling more complex analysis of the data. In some examples, the computing device(s) 12 and/or HMS 26 may apply the data to a probability model, machine learning model or other artificial intelligence developed algorithm, e.g., to determine information corresponding to patient 4 described herein. Although described in the context of examples in which IMD 10 that senses patient cardiac activity may comprise an ICM, example systems including one or more implantable, wearable, or external devices of any type configured to sense physiological parameters of a patient may be configured to implement the techniques of this disclosure.

In some examples, IMD 10 includes one or more electrodes. IMD 10 may generate electrical cardiac data (e.g., ECG data and/or EGM data) based on a cardiac signal sensed via the one or more electrodes. In some examples, the electrical cardiac data may be based on a cardiac electrical signal that indicates one or more aspects of cardiac activity of patient 4. For example, the electrical cardiac data may indicate one or more cardiac events such as atrial depolarizations (indicated by P-waves), ventricular depolarizations (indicated by R-waves), and ventricular repolarizations (indicated by T-waves). The cardiac data may represent a sequence of cardiac data points such that any given data point in the sequence of cardiac data points corresponds to a magnitude of the cardiac signal at a point in time. Based on the time at which cardiac events occur, the cardiac data may indicate one or more parameters such as heart rate and heart rate variability. IMD 10 may, in some examples, output the electrical cardiac signal to computing system 20. Computing system 20 may, in some cases, store the electrical cardiac signal to memory 24.

In some examples, memory 24 is configured to store a machine learning model. HMS 26 may be configured to apply the machine learning model to electrical cardiac data (e.g., ECG data and/or EGM data) received from IMD 10 as an input to generate an output. In some examples, the machine learning model is trained based on a plurality of sets of training data. Memory 24 may be configured to store the plurality of sets of training data. Each set of training data of the plurality of sets of training data may include a set of training electrical cardiac data and information indicating one or more values of the metric of LV dysfunction corresponding to the set of training electrical cardiac data. In some examples, the one or more values of the metric of LV dysfunction corresponding to a set of training electrical cardiac data of a set of training data may each correspond to a time relative to one or more times of the set of training electrical cardiac data. In some examples, a set of training electrical cardiac data of a set of training data may include one or more labels indicating characteristics of the set of training electrical cardiac data, but this is not required.

In some examples, electrical cardiac data received by computing system 20 from IMD 10 may include a plurality of sets electrical cardiac data. In some examples, the plurality of sets electrical cardiac data may represent segments of a long sample of electrical cardiac data. In some examples, the plurality of sets electrical cardiac data may each represent individual data samples collected by IMD 10 at different times or ranges of times. In some examples, the plurality of sets electrical cardiac data may each correspond to the same duration. In some examples, one or more sets of electrical cardiac data may correspond to a duration that is different that a duration of one or more other sets of electrical cardiac data of the plurality of sets of electrical cardiac data.

HMS 26 may apply the machine learning model to each set of electrical cardiac data of the plurality of sets of electrical cardiac data in order to determine a value of the metric of LV dysfunction corresponding to each set of electrical cardiac data of the plurality of sets of electrical cardiac data. This may allow HMS 26 to generate values of the metric of LV dysfunction corresponding to different points of time or windows of time. By determining the value of the metric of LV dysfunction corresponding to each set of electrical cardiac data of the plurality of sets of electrical cardiac data, HMS 26 may track the metric of LV dysfunction over a period of time to determine one or more trends of the metric of LV dysfunction over the period of time. In some examples, HMS 26 may determine, based on one or more determined values of the metric of LV dysfunction, a trend in the metric of LV dysfunction over a period of time. For example, HMS 26 may determine that the metric of LV dysfunction is worsening over the period of time, improving over the period of time, or remaining steady over the period of time.

IMD 10 may include a motion sensor, such as an accelerometer. The motion sensor of IMD 10 may generate motion data based on a motion of IMD 10. In some examples, IMD 10 may output the motion data to computing system 20 for analysis. HMS 26 may, in some examples, determine, based on motion data received from IMD 10, a motion value indicating an activity level of patient 4. The motion value corresponds to a value of the metric of LV dysfunction determined based on electrical cardiac data received from IMD 10. HMS 26 may determine whether to output an alert based on the motion value and the determined value of the metric of LV dysfunction. For example, HMS 26 may determine whether the value of the metric of LV dysfunction is lower than a threshold metric of LV dysfunction. HMS 26 may determine whether the motion value is greater than a threshold motion value. HMS 26 may determine whether to output the alert based on whether the value of the metric of LV dysfunction is lower than the threshold metric of LV dysfunction and whether the motion value is greater than the threshold motion value.

For example, when the metric of LV dysfunction is LVEF, it may be beneficial to output an alert when an LVEF of a patient is low and an activity level of the patient is high. In this example, HMS 26 may output an alert when the motion value is greater than the threshold motion value and when the value of the metric of LV dysfunction is lower than the threshold metric of LV dysfunction. In some examples, HMS 26 might not output an alert when the motion value is not greater than the threshold motion value or when the value of the metric of LV dysfunction is not lower than the threshold metric of LV dysfunction.

HMS 26 may train the machine learning model based on a plurality of sets of training data. By training the machine learning model based on the plurality of sets of training data, the processing circuitry is configured to cause the machine learning model to recognize one or more patterns corresponding to the metric of LV dysfunction and one or more characteristics of electrical cardiac data. Since each set of training data of the plurality of sets of training data may include a set of electrical cardiac data and one or more values of the metric of LV dysfunction corresponding to the set of training electrical cardiac data, each set of training data includes electrical cardiac data that is labeled with values of the metric of LV dysfunction. Based on characteristics of the electrical cardiac data and the labels indicating values of the metric of LV dysfunction in each set of training data, HMS 26 may train the machine learning model to recognize patterns corresponding to electrical cardiac data and the metric of LV dysfunction.

For example, once the machine learning model has been trained, HMS 26 may apply the machine learning model to a set of electrical cardiac data that indicates one or more characteristics and/or parameters (e.g., P-waves, T-waves, R-waves, heart rate, heart rate variability) in order to determine a value of the metric of LV dysfunction. HMS 26 may apply the machine learning model to process the set of electrical cardiac data to determine a value of a metric of LV dysfunction based on one or more characteristics of the set of electrical cardiac data and one or more learned patterns between characteristics of electrical cardiac signal and the metric of LV dysfunction. In some examples, the machine learning model may output an estimated real value of the metric of LV dysfunction. When the metric of LV dysfunction comprises LVEF, the real value of the metric of LV dysfunction may comprise a fraction of an amount of blood pumped out of the left ventricle to a total amount of blood in the left ventricle. In some examples, the machine learning model may output a confidence value indicating a confidence that the real value of the metric of LV dysfunction is accurate. In some examples, the machine learning model may output a confidence that the value of the metric of LV dysfunction corresponding to the set of electrical cardiac data is lower than a threshold value of the metric of LV dysfunction. In some examples, the machine learning model may output a confidence that the value of the metric of LV dysfunction corresponding to the set of electrical cardiac data is not lower than a threshold value of the metric of LV dysfunction.

In some examples, HMS 26 may label each set of training data of the plurality of sets of training data stored in memory 24. In some examples, to label each set of training data of the plurality of sets of training data, HMS 26 may identify, for each set of training data of the plurality of sets of training data, one or more characteristics of the set of training electrical cardiac data data of the set of training data. For example, HMS 26 may identify one or more P-waves, R-waves, T-waves, arrhythmia events, other electrical cardiac features, or any combination thereof. Additionally, or alternatively, HMS 26 may identify one or more parameters corresponding to the set of training electrical cardiac data such as heart rate, heart rate variability, pulse transit time (PTT) or any combination thereof. HMS 26 may label the set of training electrical cardiac data of each set of training data of the plurality of sets of training data with the one or more characteristics of the set of training electrical cardiac data.

In some examples, to label each set of training data of the plurality of sets of training data, HMS 26 may identify, in the information indicating one or more values of the metric of LV dysfunction of the set of training data, a time corresponding to each value of the one or more values of the metric of LV dysfunction. In some examples, the time corresponding to each value of the one or more values of the metric of LV dysfunction corresponds to a time that the value of the metric of LV dysfunction was measured. HMS 26 may associate the time corresponding to each value of the one or more values of the metric of LV dysfunction with a time of the set of training electrical cardiac data. That is, HMS 26 may label each set of training data of the plurality of sets of training data with one or more times corresponding to the electrical cardiac data and a time corresponding to each value of the one or more values of the metric of LV dysfunction. This means that when each set of training data of the plurality of sets of training data is labeled, the labeled training data indicates the time that values of the metric of LV dysfunction were measured relative to the times of electrical cardiac data.

The techniques described herein are not limited to examples where IMD 10 comprises an ICM. In some examples, IMD 10 of FIG. 1 may include any kind of external or implantable medical device that is configured to collect electrical cardiac data such as ECG data or EGM data. For example, IMD 10 may include an implantable cardioverter-defibrillator (ICD), a pacemaker, a cardiac resynchronization therapy pacemaker (CRT-P), a cardiac resynchronization therapy defibrillator (CRT-D), or an external cardiac signal sensing device.

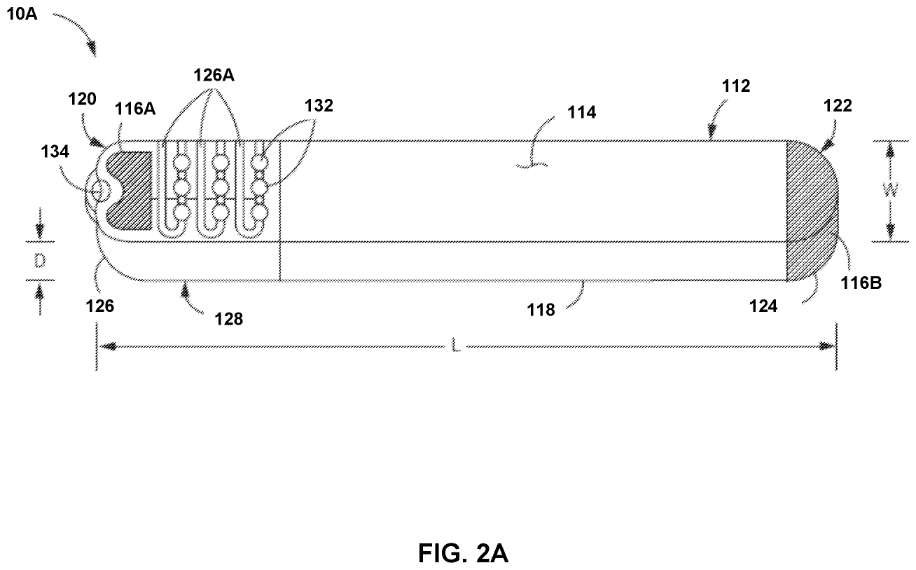

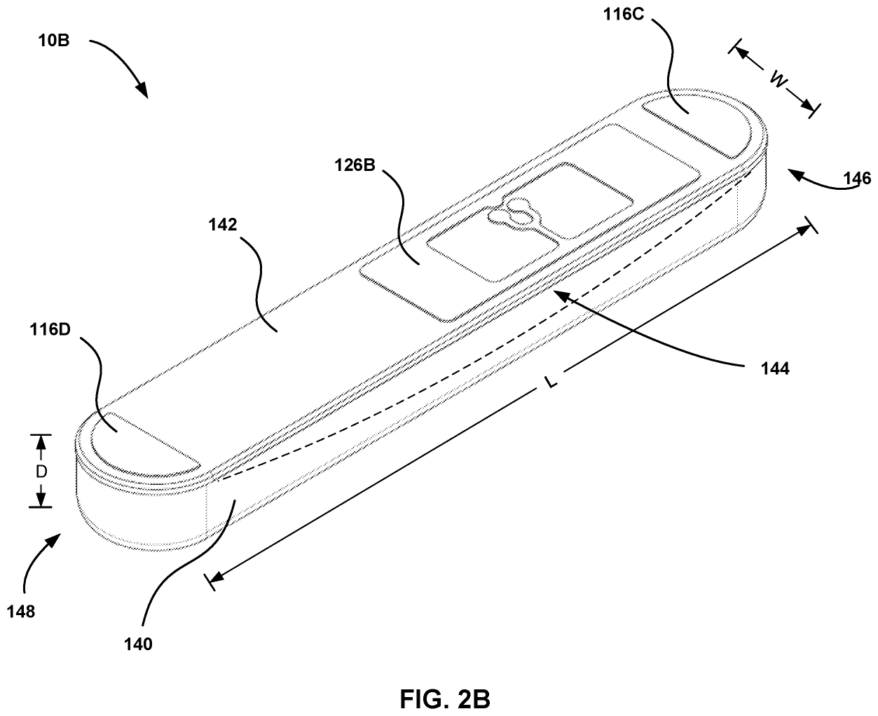

FIG. 2A is a perspective drawing illustrating an IMD 10A, which may be an example configuration of IMD 10 of FIG. 1 as an ICM, in accordance with one or more techniques of this disclosure. In the example shown in FIG. 2A, IMD 10A may be embodied as a monitoring device having housing 112, proximal electrode 116A and distal electrode 116B. Housing 112 may further comprise first major surface 114, second major surface 118, proximal end 120, and distal end 122. Housing 112 encloses electronic circuitry located inside the IMD 10A and protects the circuitry contained therein from body fluids. Housing 112 may be hermetically sealed and configured for subcutaneous implantation. Electrical feedthroughs provide electrical connection of electrodes 116A and 116B.

In the example shown in FIG. 2A, IMD 10A is defined by a length L, a width Wand thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 116A and distal electrode 116B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of first major surface 114 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within the patient, the first major surface 114 faces outward, toward the skin of the patient while the second major surface 118 is located opposite the first major surface 114. In addition, in the example shown in FIG. 2A, proximal end 120 and distal end 122 are rounded to reduce discomfort and irritation to surrounding tissue once inserted under the skin of the patient. IMD 10A, including instrument and method for inserting IMD 10A is described, for example, in U.S. Pat. No. 11,311,312, incorporated herein by reference in its entirety.

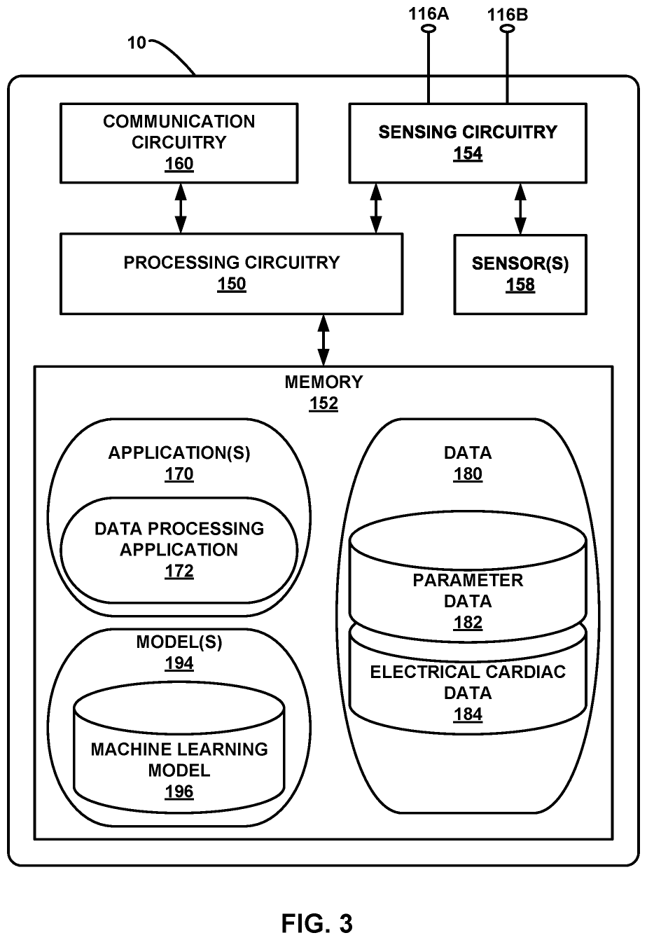

Proximal electrode 116A is at or proximate to proximal end 120, and distal electrode 116B is at or proximate to distal end 122. Proximal electrode 116A and distal electrode 116B are used to sense electrical cardiac signals, e.g., ECG signals and/or EGM signals, and measure interstitial impedance thoracically outside the ribcage, which may be sub-muscularly or subcutaneously. Electrical cardiac signals and impedance measurements may be stored in a memory of IMD 10A, and data may be transmitted via integrated antenna 126A to another device, which may be another implantable device or an external device, such as computing device 12. In some example, electrodes 116A and 116B may additionally or alternatively be used for sensing any bio-potential signal of interest, which may be, for example, EGM, electroencephalogram (EEG), electromyogram (EMG), or a nerve signal, from any implanted location. Housing 112 may house the circuitry of IMD 10 illustrated in FIG. 3.

In the example shown in FIG. 2A, proximal electrode 116A is at or in close proximity to the proximal end 120 and distal electrode 116B is at or in close proximity to distal end 122. In this example, distal electrode 116B is not limited to a flattened, outward facing surface, but may extend from first major surface 114 around rounded edges 124 and/or end surface 126 and onto the second major surface 118 so that the electrode 116B has a three-dimensional curved configuration. In some examples, electrode 116B is an uninsulated portion of a metallic, e.g., titanium, part of housing 112.

In the example shown in FIG. 2A, proximal electrode 116A is located on first major surface 114 and is substantially flat, and outward facing. However, in other examples proximal electrode 116A may utilize the three-dimensional curved configuration of distal electrode 116B, providing a three-dimensional proximal electrode (not shown in this example). Similarly, in other examples distal electrode 116B may utilize a substantially flat, outward facing electrode located on first major surface 114 similar to that shown with respect to proximal electrode 116A.

The various electrode configurations allow for configurations in which proximal electrode 116A and distal electrode 116B are located on both first major surface 114 and second major surface 118. In other configurations, such as that shown in FIG. 2A, only one of proximal electrode 116A and distal electrode 116B is located on both first major surface 114 and second major surface 118, and in still other configurations both proximal electrode 116A and distal electrode 116B are located on one of the first major surface 114 or the second major surface 118 (e.g., proximal electrode 116A located on first major surface 114 while distal electrode 116B is located on second major surface 118). In another example, IMD 10A may include electrodes on both first major surface 114 and second major surface 118 at or near the proximal and distal ends of the device, such that a total of four electrodes are included on IMD 10A. Electrodes 116A and 116B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 120 includes a header assembly 128 that includes one or more of proximal electrode 116A, integrated antenna 126A, anti-migration projections 132, and/or suture hole 134. Integrated antenna 126A is located on the same major surface (i.e., first major surface 114) as proximal electrode 116A and is also included as part of header assembly 128. Integrated antenna 126A allows IMD 10A to transmit and/or receive data. In other examples, integrated antenna 126A may be formed on the opposite major surface as proximal electrode 116A, or may be incorporated within the housing 112 of IMD 10A. In the example shown in FIG. 2A, anti-migration projections 132 are located adjacent to integrated antenna 126A and protrude away from first major surface 114 to prevent longitudinal movement of the device. In the example shown in FIG. 2A, anti-migration projections 132 include a plurality (e.g., nine) small bumps or protrusions extending away from first major surface 114. As discussed above, in other examples anti-migration projections 132 may be located on the opposite major surface as proximal electrode 116A and/or integrated antenna 126A. In addition, in the example shown in FIG. 2A, header assembly 128 includes suture hole 134, which provides another means of securing IMD 10A to the patient to prevent movement following insertion. In the example shown, suture hole 134 is located adjacent to proximal electrode 116A. In one example, header assembly 128 is a molded header assembly made from a polymeric or plastic material, which may be integrated or separable from the main portion of IMD 10A.

In some examples, IMD 10A may be configured to collect an electrical cardiac signal such as an ECG signal or an EGM signal via electrodes 116A, 116B. In some examples, the electrical cardiac signal collected by IMD 10A may include one or more characteristics and/or indicate one or more patient parameters. IMD 10A may be configured to process the electrical cardiac signal, pre-process the electrical cardiac signal, output the electrical cardiac signal, store the electrical cardiac signal in a memory, or any combination thereof.

FIG. 2B is a perspective drawing illustrating another IMD 10B, which may be another example configuration of IMD 10 from FIG. 1 as an ICM, in accordance with one or more techniques of this disclosure. IMD 10B of FIG. 2B may be configured substantially similarly to IMD 10A of FIG. 2A, with differences between them discussed herein.

IMD 10B may include a leadless, subcutaneously implantable monitoring device, e.g., an ICM. IMD 10B includes housing having a base 140 and an insulative cover 142. Proximal electrode 116C and distal electrode 116D may be formed or placed on an outer surface of insulative cover 142. Various circuitries and components of IMD 10B, e.g., described with respect to FIG. 3, may be formed or placed on an inner surface of insulative cover 142, or within base 140. In some examples, a battery or other power source of IMD 10B may be included within base 140. In the illustrated example, antenna 126B is formed or placed on the outer surface of insulative cover 142, but may be formed or placed on the inner surface in some examples. In some examples, insulative cover 142 may be positioned over an open base 140 such that base 140 and insulative cover 142 enclose the circuitries and other components and protect them from fluids such as body fluids. The housing including base 140 and insulative cover 142 may be hermetically sealed and configured for subcutaneous implantation.

Circuitries and components may be formed on the inner side of insulative cover 142, such as by using flip-chip technology. Insulative cover 142 may be flipped onto a base 140. When flipped and placed onto base 140, the components of IMD 10B formed on the inner side of insulative cover 142 may be positioned in a gap 144 defined by base 140. Electrodes 116C and 116D and antenna 126B may be electrically connected to circuitry formed on the inner side of insulative cover 142 through one or more vias (not shown) formed through insulative cover 142. Insulative cover 142 may be formed of sapphire (i.e., corundum), glass, parylene, and/or any other suitable insulating material. Base 140 may be formed from titanium or any other suitable material (e.g., a biocompatible material). Electrodes 116C and 116D may be formed from any of stainless steel, titanium, platinum, iridium, or alloys thereof. In addition, electrodes 116C and 116D may be coated with a material such as titanium nitride or fractal titanium nitride, although other suitable materials and coatings for such electrodes may be used.

In the example shown in FIG. 2B, the housing of IMD 10B defines a length L, a width Wand thickness or depth D and is in the form of an elongated rectangular prism wherein the length L is much larger than the width W, which in turn is larger than the depth D, similar to IMD 10A of FIG. 2A. For example, the spacing between proximal electrode 116C and distal electrode 116D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of insulative cover 142 faces outward, toward the skin of the patient. In addition, as shown in FIG. 2B, proximal end 146 and distal end 148 are rounded to reduce discomfort and irritation to surrounding tissue once inserted under the skin of the patient. In addition, edges of IMD 10B may be rounded.

In some examples, IMD 10B may be configured to collect an electrical cardiac signal such as an ECG signal or an EGM signal via electrodes 116C, 116D. In some examples, the electrical cardiac signal collected by IMD 10B may include one or more characteristics and/or indicate one or more patient parameters. IMD 10B may be configured to process the electrical cardiac signal, pre-process the electrical cardiac signal, output the electrical cardiac signal, store the electrical cardiac signal in a memory, or any combination thereof.

FIG. 3 is a block diagram illustrating an example configuration of IMD 10 of FIG. 1, in accordance with one or more techniques of this disclosure. As shown in FIG. 3, IMD 10 includes processing circuitry 150, memory 152, sensing circuitry 154 coupled to electrodes 116A and 116B (hereinafter, “electrodes 116A, 116B”) and one or more sensor(s) 158, and communication circuitry 160.

Processing circuitry 150 may include fixed function circuitry and/or programmable processing circuitry. Processing circuitry 150 may include any one or more of a microprocessor, a controller, a GPU, a TPU, a DSP, an ASIC, a FPGA, or equivalent discrete or analog logic circuitry. In some examples, processing circuitry 150 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 150 herein may be embodied as software, firmware, hardware, or any combination thereof. In some examples, memory 152 includes computer-readable instructions that, when executed by processing circuitry 150, cause IMD 10 and processing circuitry 150 to perform various functions attributed herein to IMD 10 and processing circuitry 150. Memory 152 may include any volatile, non-volatile, magnetic, optical, or electrical media, such as a RAM, ROM, NVRAM, EEPROM, flash memory, or any other digital media.

Sensing circuitry 154 may sense a cardiac signal and measure impedance, e.g., of tissue proximate to IMD 10, via electrodes 116A, 116B. The measured impedance may vary based on respiration, cardiac pulse or flow, and a degree of perfusion or edema. Processing circuitry 150 may determine patient metrics relating to respiration, fluid retention, cardiac pulse or flow, perfusion, and/or edema based on the measured impedance. In some examples, processing circuitry 150 may identify features of the sensed cardiac signal, such as heart rate, heart rate variability, T-wave alternans, intra-beat intervals (e.g., QT intervals), and/or morphologic features, to detect an episode of cardiac arrhythmia of patient 4.

In some examples, IMD 10 includes one or more sensors 158, such as one or more accelerometers, gyroscopes, microphones, optical sensors, temperature sensors, pressure sensors, and/or chemical sensors. In some examples, sensing circuitry 154 may include one or more filters and amplifiers for filtering and amplifying signals received from one or more of electrodes 116A, 116B and/or sensors 158. In some examples, sensing circuitry 154 and/or processing circuitry 150 may include a rectifier, filter and/or amplifier, a sense amplifier, comparator, and/or analog-to-digital converter. Processing circuitry 150 may determine parameter data 182, e.g., values of physiological parameters of patient 4, based on signals from sensors 158, which may be stored as data 180 in memory 152. Patient parameters determined from signals from sensors 158 may include intravascular fluid level, interstitial fluid level, oxygen saturation, glucose level, stress hormone level, heart sounds, body motion, activity intensity, sleep duration, sleep quality, body posture, or blood pressure.

In addition to data 180, memory 152 may store applications 170 executable by processing circuitry 150. Applications 170 may include a data processing application 172. Processing circuitry 150 may execute data processing application 172 to process parameter data 182 and electrical cardiac data 184. In some examples, data processing application 172 may identify information corresponding to parameter data 182 and/or electrical cardiac data 184. For example, data processing application 172 may identify a heart rate of patient 4 based on electrical cardiac data 184 by identifying a rate at which R-waves occur in electrical cardiac data 184.

Data 180 may include electrical cardiac data 184 sensed by IMD 10 via electrodes 116A, 116B. In some examples, electrical cardiac data 184 may represent cardiac data such as ECG data or EGM data that indicates cardiac activity of patient 4. In some examples, electrical cardiac data 184 may indicate cardiac activity of patient 4 over a long period of time (e.g., weeks or months) continuously collected via electrodes 116A, 116B while IMD 10 is implanted underneath the skin of patient 4. In some examples, electrical cardiac data 184 may include a plurality of sets of cardiac data each collected via electrodes 116A, 116B when IMD 10 is implanted underneath the skin of patient 4. Electrical cardiac data 184 may include a data indicating a cardiac signal, heart rate information, R-R interval information, morphological information, or other information.

Processing circuitry 150 may communicate parameter data 182 and electrical cardiac data 184 to one or more other computing devices, e.g., computing device(s) 12 and/or computing system 20, using communication circuitry 160. Communication circuitry 160 may include any suitable hardware, firmware, software or any combination thereof for wirelessly communicating with another device. Communication circuitry 160 may be configured to transmit and/or receive signals via inductive coupling, electromagnetic coupling, Near Field Communication (NFC), Radio Frequency (RF) communication, Bluetooth®, Wi-Fi, or other proprietary or non-proprietary wireless communication schemes.

In some examples, memory 152 of IMD 10 is configured to store model(s) 194 including a machine learning model 196. IMD 10 may be configured to apply machine learning model 196 to electrical cardiac data 184 in order to determine a value of a metric of LV dysfunction corresponding to electrical cardiac data 184. In some examples, machine learning model 196 may be trained by computing system 20 of FIG. 1 and output to IMD 10. IMD 10 is not required to store model(s) 194 including a machine learning model 196. IMD 10 may, in some examples, output data 180 to one or more other devices (e.g., external device 12, computing system 20, clinician computing devices 38, or any combination thereof) for processing.

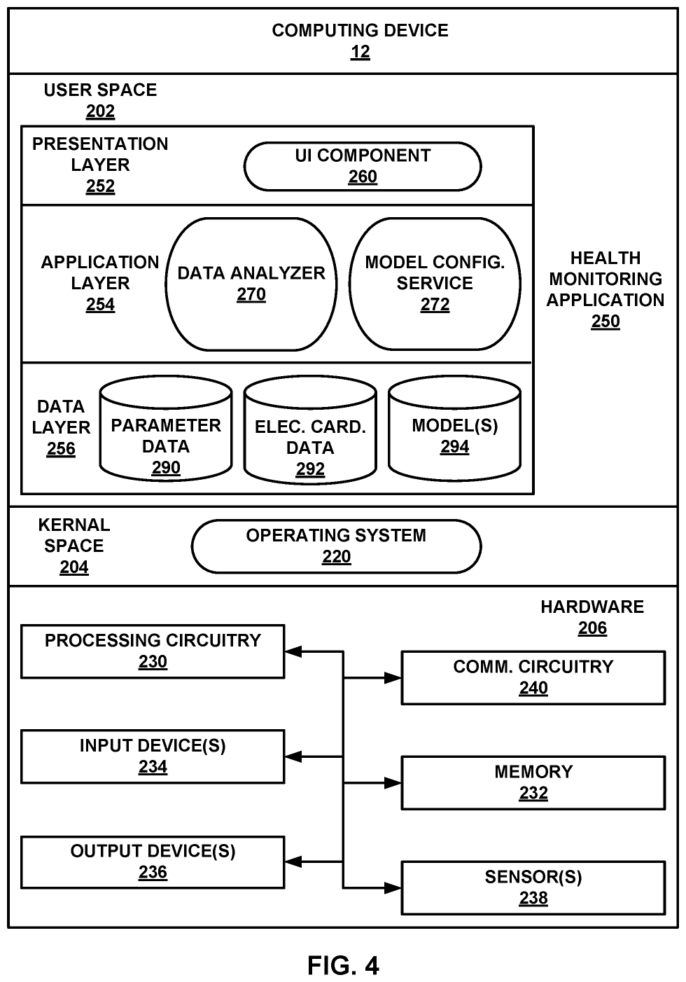

FIG. 4 is a block diagram illustrating an example configuration of a computing device 12, which may correspond to either (or both operating in coordination) of computing devices 12A and 12B, in accordance with one or more techniques of this disclosure. In some examples, computing device 12 takes the form of a smartphone, a laptop, a tablet computer, a personal digital assistant (PDA), a smartwatch or other wearable computing device. In some examples, IoT devices 30 and/or clinician computing devices 38 may be configured similarly to the configuration of computing device 12 illustrated in FIG. 4.

As shown in the example of FIG. 4, computing device 12 may be logically divided into user space 202, kernel space 204, and hardware 206. Hardware 206 may include one or more hardware components that provide an operating environment for components executing in user space 202 and kernel space 204. User space 202 and kernel space 204 may represent different sections or segmentations of memory, where kernel space 204 provides higher privileges to processes and threads than user space 202. For instance, kernel space 204 may include operating system 220, which operates with higher privileges than components executing in user space 202.

As shown in FIG. 4, hardware 206 includes processing circuitry 230, memory 232, one or more input device(s) 234, one or more output device(s) 236, one or more sensor(s) 238, and communication circuitry 240. Although shown in FIG. 4 as a stand-alone device for purposes of example, computing device 12 may be any component or system that includes processing circuitry or other suitable computing environment for executing software instructions and, for example, need not necessarily include one or more elements shown in FIG. 4.

Processing circuitry 230 is configured to implement functionality and/or process instructions for execution within computing device 12. For example, processing circuitry 230 may be configured to receive and process instructions stored in memory 232 that provide functionality of components included in kernel space 204 and user space 202 to perform one or more operations in accordance with techniques of this disclosure. Examples of processing circuitry 230 may include, any one or more microprocessors, controllers, GPUs, TPUs, DSPs, ASICs, FPGAs, or equivalent discrete or integrated logic circuitry.

Memory 232 may be configured to store information within computing device 12, for processing during operation of computing device 12. Memory 232, in some examples, is described as a computer-readable storage medium. In some examples, memory 232 includes a temporary memory or a volatile memory. Examples of volatile memories include RAM, DRAM, static random access memory (SRAM), and other forms of volatile memories known in the art. Memory 232, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or EEPROM memories. In some examples, memory 232 includes cloud-associated storage.

One or more input device(s) 234 of computing device 12 may receive input, e.g., from patient 4, clinicians 40, or another user. Examples of input are tactile, audio, kinetic, and optical input. Input device(s) 234 may include, as examples, a mouse, keyboard, voice responsive system, camera, buttons, control pad, microphone, presence-sensitive or touch-sensitive component (e.g., screen), or any other device for detecting input from a user or a machine.

One or more output device(s) 236 of computing device 12 may generate output, e.g., to patient 4 or another user. Examples of output are tactile, haptic, audio, and visual output. Output device(s) 236 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensor(s) 238 of computing device 12 may sense physiological parameters or signals of patient 4. Sensor(s) 238 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an analog-to-digital converter (ADC)), similar to those described above with respect to IMDs 10 and FIG. 3.

Communication circuitry 240 of computing device 12 may communicate with other devices by transmitting and receiving data. Communication circuitry 240 may receive data from IMD 10, such as patients metrics and/or higher resolution diagnostic information, from communication circuitry in IMD 10. Communication circuitry 240 may include a network interface card, such as an Ethernet card, an optical transceiver, a radio frequency transceiver, or any other type of device that can send and receive information. For example, communication circuitry 160 may include a radio transceiver configured for communication according to standards or protocols, such as 3G, 4G, 5G, Wi-Fi (e.g., 802.11 or 802.15 ZigBee), Bluetooth®, or BLE.

As shown in FIG. 4, health monitoring application 250 executes in user space 202 of computing device 12. Health monitoring application 250 may be logically divided into presentation layer 252, application layer 254, and data layer 256. Presentation layer 252 may include a user interface (UI) component 260, which generates and renders user interfaces of health monitoring application 250.

Data layer 256 may include parameter data 290 and electrical cardiac data 292, which may be received from IMD 10 via communication circuitry 240, and stored in memory 232 by processing circuitry 230. Application layer 254 may include, but is not limited to, data analyzer 270, and model configuration service 272. Data analyzer 270 may be configured to process parameter data 290 and/or electrical cardiac data 292 generated by IMD 10 to generate information corresponding to one or more patient conditions of patient 4. Data analyzer 270 may determine the information corresponding to patient 4 based on application of parameter data 290 and/or electrical cardiac data 292 as inputs one or more model(s) 294, which may include one or more probability models, machine learning models, algorithms, decision trees, and/or thresholds. In examples in which model(s) 294 include one or more machine learning models, data analyzer 270 may apply feature vectors derived from the data to the model(s) 294.

Model configuration service 272 may be configured to modify model(s) 294 based on feedback indicating whether determinations were accurate or updated parameters received, e.g., from HMS 26. In some examples, model configuration service 272 may utilize the data sets from patient 4 for supervised machine learning to further train models included as part of model(s) 294. Model configuration service 272, or another component executed by processing circuitry of medical device system 2, may select a configuration of model(s) 294 based on etiological data for patient. In some examples, different model(s) 294 tailored to different cohorts of patients may be available for selection for patient 4 based on such etiological data.

In some examples, model(s) 294 include a machine learning model that is configured to process electrical cardiac data 292 to determine a value of a metric of LV dysfunction. Computing device 12 may be configured to apply the machine learning model to electrical cardiac data 292 in order to determine a value of a metric of LV dysfunction corresponding to electrical cardiac data 292. In some examples, the machine learning model may be trained by computing system 20 of FIG. 1 and output computing device 12. Computing device 12 is not required to store model(s) 294 including the machine learning model. Computing device 12 may, in some examples, output electrical cardiac data 292 to one or more other devices (e.g., computing system 20, clinician computing devices 38, or any combination thereof) for processing and/or receive one or more values determined based on processing electrical cardiac data 292.

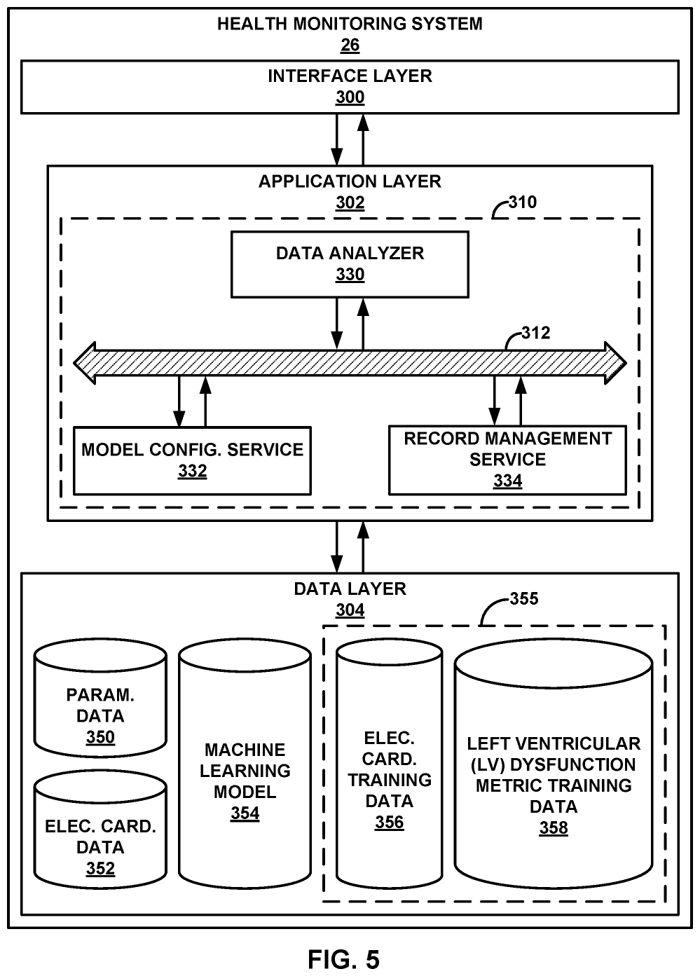

FIG. 5 is a block diagram illustrating an operating perspective of HMS 26, in accordance with one or more techniques of this disclosure. HMS 26 may be implemented in a computing system 20, which may include hardware components such as processing circuitry 22, memory 24, and communication circuitry, embodied in one or more physical devices. FIG. 5 provides an operating perspective of HMS 26 when hosted as a cloud-based platform. In the example of FIG. 5, components of HMS 26 are arranged according to multiple logical layers that implement the techniques of this disclosure. Each layer may be implemented by one or more modules comprised of hardware, software, or a combination of hardware and software.

Computing devices, such as computing device(s) 12, IoT devices 30, and clinician computing devices 38 operate as clients that communicate with HMS 26 via interface layer 300. The computing devices typically execute client software applications, such as desktop application, mobile application, and web applications. Interface layer 300 represents a set of application programming interfaces (API) or protocol interfaces presented and supported by HMS 26 for the client software applications. Interface layer 300 may be implemented with one or more web servers.

As shown in FIG. 5, HMS 26 also includes an application layer 302 that represents a collection of services 310 for implementing the functionality ascribed to HMS 26 herein. Application layer 302 receives information from client applications, e.g., data from a computing device 12 or IoT device 30 (some or all of which may have been retrieved from IMD 10), and further processes the information according to one or more of the services 310 to respond to the information. Application layer 302 may be implemented as one or more discrete software services 310 executing on one or more application servers, e.g., physical or virtual machines. That is, the application servers provide runtime environments for execution of services 310. In some examples, the functionality interface layer 300 as described above and the functionality of application layer 302 may be implemented at the same server. Services 310 may communicate via a logical service bus 312. Service bus 312 generally represents a logical interconnection or set of interfaces that allows different services 310 to send messages to other services, such as by a publish/subscription communication model.

Data layer 304 of HMS 26 provides persistence for information in HMS 26 using one or more data repositories. A data repository, generally, may be any data structure or software that stores and/or manages data. Examples of data repositories include but are not limited to relational databases, multi-dimensional databases, maps, and hash tables, to name only a few examples.

Services 310 may include data analyzer 330, model configuration service 332, and record management service 334. As shown in FIG. 5, each of services 310 is implemented in a modular form within HMS 26. Although shown as separate modules for each service, in some examples the functionality of two or more services may be combined into a single module or component. Each of services 310 may be implemented in software, hardware, or a combination of hardware and software. Moreover, services 310 may be implemented as standalone devices, separate virtual machines or containers, processes, threads or software instructions generally for execution on one or more physical processors. Record management service 334 may store received patient data as parameter data 350 and electrical cardiac data 352.

Data analyzer 330 may determine information corresponding to cardiac activity of patient 4 based on electrical cardiac data 352, and in some cases other parameter data 350, generated by IMD 10. In some examples, data analyzer 330 may identify one or more features (e.g., R-waves, T-waves, P-waves) in electrical cardiac data 352 received from IMD 10. In some examples, data analyzer 330 may determine whether electrical cardiac data 352 indicates a patient condition or arrythmia such HF, VF, AF or AV block. Data analyzer 330 may determine the information corresponding to patient 4 based on application of parameter data 350 and/or electrical cardiac data 352 as inputs to machine learning model 354. In some examples, data analyzer 330 may apply feature vectors derived from the data to machine learning model 354.

Machine learning model 354 may be developed by model configuration service 332. Example machine learning techniques that may be employed to generate machine learning model 354 include various learning styles, such as supervised learning, unsupervised learning, and semi-supervised learning. Example types of algorithms include Bayesian algorithms, Markov models, Hawkes processes, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, self-attention models, Convolutional Neural Networks (CNNs), Long Short Term Networks (LSTMs), the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, model configuration service 332 may be configured to train machine learning model 354 using training data 355. By training the machine learning model based on training data 355, model configuration service 332 is configured to cause machine learning model 354 to recognize one or more patterns corresponding to a metric of LV dysfunction (e.g., LVEF) and one or more characteristics of electrical cardiac data. For example, model configuration service 332 is configured to cause machine learning model 354 to recognize one or more patterns in electrical cardiac data corresponding to one or more values of the metric of LV dysfunction. This may allow HMS 26 may use the machine learning model 354 to process incoming samples of electrical cardiac training data to determine values of the metric of LV dysfunction.

Training data 355 may include electrical cardiac training data 356 and LV dysfunction metric training data 358. In some examples, training data 355 may include a plurality of sets of training data, where each set of training data of the plurality of sets of training data includes a set of electrical cardiac training data of electrical cardiac training data 356 and a set of LV dysfunction metric training data of LV dysfunction metric training data 358. In some examples, the set of electrical cardiac training data and the set of LV dysfunction metric training data of a set of training data may correspond to the same patient. That is, the set of electrical cardiac training data may be collected from the patient and the set of LV dysfunction metric training data may include one or more values of the metric of LV dysfunction measured from the patient.

To train machine learning model 354, model configuration service 332 may label and/or classify each set of training data of the plurality of sets of training data of training data 355. In some examples, to label each set of training data of training data 355, model configuration service 332 may label the set of electrical cardiac training data of each set of training data with one or more characteristics, fiducials, and/or features of the set of electrical cardiac training data. In some examples, to label each set of training data of training data 355, machine learning model 354 may label the set of electrical cardiac training data of each set of training data with one or more parameters corresponding to the set of electrical cardiac training data such as heart rate, heart rate variability, pulse transit time, or any combination thereof. In some examples, to label each set of training data of training data 355, machine learning model 354 may label the set of electrical cardiac training data of each set of training data with one or more arrythmias or patient conditions corresponding to the set of electrical cardiac training data such as HF, AV, AF, AV block, or any combination thereof. In some examples, data analyzer 330 may identify one or more characteristics, fiducials, features, parameters, patient conditions, arrythmias, or any combination thereof corresponding to each set of electrical cardiac training data of electrical cardiac training data 356.

In some examples, to label each set of training data of training data 355, model configuration service 332 may identify, in the set of LV dysfunction metric training data of LV dysfunction metric training data 358 corresponding to each set of training data of training data 355, one or more values of the metric of LV dysfunction. In some examples, each value of the one or more values of the metric of LV dysfunction may correspond to a time or a range of times at which the value of the metric of LV dysfunction was measured from a patient. Model configuration service 332 may associate the time corresponding to each value of the one or more values of the metric of LV dysfunction of the set of LV dysfunction metric training data with a time or range of times of the corresponding set of electrical cardiac training data. For example, since each set of training data of training data 355 includes a set of electrical cardiac training data of electrical cardiac training data 356 and a set of LV dysfunction metric training data of LV dysfunction metric training data 358 collected from the same patient, model configuration service 332 may associate times at which the electrical cardiac training data was collected from the patient with the time at which each value of the one or more values of the metric of LV dysfunction was measured from the patient. This may allow model configuration service 332 to identify patterns in electrical cardiac training data that indicate values of the metric of LV dysfunction.

Model configuration service 332 may, in some examples, create a plurality of sets of training data 355 that each include a set of electrical cardiac training data (e.g., ECG data and/or EGM data) and a set of LV dysfunction metric training data. In some examples, the set of electrical cardiac training data and the set of LV dysfunction metric training data may be collected from the same patient at different times. For example, the set of electrical cardiac training data and the set of LV dysfunction metric training data may be collected from the same patient less than one day apart, less than one week apart, less than two weeks apart, or another amount of time apart. In some examples, the set of electrical cardiac training data and the set of LV dysfunction metric training data may be collected from the same patient at the same time and/or during overlapping windows of time. In any case, Model configuration service 332 may associate, for each set of training data of training data 355, a time or range of times corresponding to a respective set of electrical cardiac training data of electrical cardiac training data 356 with times or ranges of times corresponding to a respective set of LV dysfunction metric training data of LV dysfunction metric training data 358. This may allow model configuration service 332 to train machine learning model 354 to recognize patterns in electrical cardiac data that indicate values of the metric of LV dysfunction.

In some examples, electrical cardiac training data 356 may include a plurality of sets of electrical cardiac training data. The plurality of sets of electrical cardiac data may include one or more electrical cardiac measurements each collected from a human patient via a Holter monitor, one or more sets of electrical cardiac data collected via electrodes attached to skin of human patients, one or more sets of electrical cardiac data collected from patients by wearable devices (e.g., smart watches), one or more sets of electrical cardiac data collected from human patients via IMDs, or any combination thereof. In any case, each set of electrical cardiac data of the plurality of sets of electrical cardiac training data within electrical cardiac training data 356 may include electrical data that indicates cardiac activity of a patient, such as one or more cardiac cycles of the myocardium of the patient.

Model configuration service 332 may, in some examples, train machine learning model 354 based on one or more sets of raw electrical cardiac training data of electrical cardiac training data 356. Raw electrical cardiac training data may indicate one or more features, characteristics, and/or parameters of cardiac activity without including labels identifying those features or characteristics. For example, a set of raw electrical cardiac training data may indicate one or more R-waves, T-waves, P-waves, and other features or characteristics without including labels a set of raw electrical cardiac training data may indicate identifying those characteristics. Additionally, or alternatively, a set of raw electrical cardiac training data may indicate parameters such as heart rate and/or heart rate variability without including labels identifying values of these parameters.

Model configuration service 332 may, in some examples, train machine learning model 354 based on one or more labeled sets of raw electrical cardiac training data of electrical cardiac training data 356. Labeled electrical cardiac training data may include labels identifying one or more features, characteristics, and/or parameters of cardiac activity indicated by electrical cardiac training data. For example, a set of labeled electrical cardiac training data may include one or more labels identifying one or more R-waves, T-waves, P-waves, and other features or characteristics. Additionally, or alternatively, a set of labeled electrical cardiac training data may include one or more labels identifying parameter values such as heart rate and/or heart rate variability corresponding to the labeled electrical cardiac training data.

In some examples, to train machine learning model 354, it may be beneficial for model configuration service to use information corresponding to training data in order to associate patterns and aspects of training data with known characteristics of the training data. For example, when model configuration service 332 is training machine learning model 354 to identify fiducials in cardiac signals such as ECG signals and EGM signals, it may be beneficial for the training data to include a plurality of cardiac data samples having labels that identify fiducials (e.g., labels identifying P-waves, R-waves, T-waves and other fiducials). Additionally, or alternatively, when model configuration service 332 is training machine learning model 354 to identify arrythmias such as AF or AV block in cardiac data, it may be beneficial for the training data to include one or more labels indicating portions of the training data that indicate arrythmia.

In some examples, model configuration service 332 may train machine learning model 354 to process electrical cardiac data to determine a value of LVEF. In some examples, LVEF may represent a ratio of a volume of blood ejected from the left ventricle in response to ventricular depolarization to a volume of blood present in the left ventricle immediately prior to ventricular depolarization. LVEF may be expressed as a percentage (e.g., 50% of the blood present in the left ventricle before depolarization was ejected from the ventricle in response to depolarization). In some examples, machine learning model 354 may represent a regression model when machine learning model 354 outputs a percentage and/or a ratio value of LVEF.

In some examples, model configuration service 332 may represent a classification model when machine learning model 354 outputs a confidence that LVEF of a patient is low (e.g., <35%). For example, machine learning model 354 may process electrical cardiac data 352 and output a confidence that a value of a metric of LV dysfunction (e.g., a value of LVEF) is less than a threshold value of a metric of LV dysfunction. In some examples, the confidence output by machine learning model 354 may represent a probability within a range between 0 and 1 that the electrical cardiac data 352 corresponding to a patient indicates that the patient is associated with a value of a metric of LV dysfunction lower than the threshold value of the metric of LV dysfunction. In some examples, machine learning model 354 may process electrical cardiac data 352 and output a confidence that a value of a metric of LV dysfunction (e.g., a value of LVEF) is not less than a threshold value of a metric of LV dysfunction.

Machine learning model 354 may be configured to process electrical cardiac data of electrical cardiac data 352 from any patient do determine a value of a metric of LV dysfunction. For example, machine learning model 354 may process a set of electrical cardiac data corresponding to a patient at rest to determine a value of a metric of LV dysfunction. In other examples, machine learning model 354 may process a set of electrical cardiac data corresponding to an active patient to determine a value of a metric of LV dysfunction. In other examples, machine learning model 354 may process a set of electrical cardiac data corresponding to a patient exhibiting signs of heart failure to determine a value of a metric of LV dysfunction. Machine learning model 354 may process a set of electrical cardiac data corresponding to a patient under any conditions to determine a value of a metric of LV dysfunction.

In some examples, model configuration service 332 may use paired electrical cardiac training data 356 and LV dysfunction metric training data 358 (e.g., point-in-time ECG-LVEF paired data) to develop machine learning model 354 as a classification model to determine whether a value of a metric of LV dysfunction is low or high (e.g., LVEF<35%). In some examples, model configuration service 332 may use paired electrical cardiac training data 356 and LV dysfunction metric training data 358 (e.g., point-in-time ECG-LVEF paired data) to develop machine learning model 354 as a regression model to estimate a precise value of a metric of LV dysfunction (e.g., LVEF=64%). When machine learning model 354 is a classification model, machine learning model 354 may output a confidence that a value of a metric of LV dysfunction is lower and/or higher than a threshold. When machine learning model 354 is a regression model, machine learning model 354 may output a precise estimate of a value of a metric of LV dysfunction.

Model configuration service 332 may, in some cases, collect longitudinal electrical cardiac training data (e.g., longitudinal ECG data) and point-in-time pairing of electrical cardiac data and LV dysfunction metric data, and longitudinal LV dysfunction metric data (e.g., longitudinal EF data) to develop machine learning model 354 as a classification model and/or a regression model. In one example, machine learning model 354 may pair a set of ECG data of electrical cardiac training data 356 at times t₁, t₂, . . . tₙ (e.g., ECG(t₁), ECG(t₂), . . . ECG(tₙ)) with a set of LVEF data of LV dysfunction metric training data 358 at time tₘ is (e.g., LVEF(tₘ)) to create a set of training data of training data 355. Paired sets of electrical cardiac training data and LV dysfunction metric training data may correspond to different points in time, the same point in time, overlapping points of time and/or opening windows of time, or any combination thereof.

In some examples, model configuration service 332 may train machine learning model 354 to be a classification model configured to output a confidence that LVEF is below a threshold value of LVEF. In some examples, the threshold value of LVEF is 35%, but this is not required. The threshold value of LVEF may be any value. In some examples, when LVEF is below the threshold value of LVEF, this may indicate that a patient is at risk of experiencing one or more conditions such as heart failure. In some examples, LVEF of a patient may vary depending one or more factors such as activity level, posture, whether the patient is awake or asleep, among other factors.

When model configuration service 332 trains machine learning model 354 to be a classification model, model configuration service 332 may use point-in-time paired sets of electrical cardiac training data and LV dysfunction metric training data. That is, model configuration service 332 may train machine learning model 354 using a plurality of sets of training data of training data 355 that each include a set of electrical cardiac training data of electrical cardiac training data 356 and a set of LV dysfunction metric training data of LV dysfunction metric training data 358 collected from a patient at the same point in time, during the same window of time, or during overlapping windows of time. When model configuration service 332 trains machine learning model 354 to be a classification model configured to output a confidence that LVEF is below a threshold value of LVEF using point-in-time paired training data, machine learning model 354 may process electrical cardiac data of electrical cardiac data 352 collected from a patient and output a confidence that LVEF of the patient is below a threshold value of LVEF at a time or window of time that the electrical cardiac data is collected from the patient.

In some examples, machine learning model 354 may accept a set of ECG data of electrical cardiac data 352 corresponding to time k as an input, and output an estimated value of LVEF at time m. The machine learning model 354 may operate according to the following equation:

$$EF ( {t}_{m} ) : F ( ECG ( {t}_{k} ) ) ∼ = EF ( {t}_{m} ) , \qquad ( eq . 1 )$$

$$when ( m - k ) < 1 week$$

As seen above in equation 1, machine learning model 354 may accept a set of ECG data of electrical cardiac data 352 corresponding to time k as an input, and output an estimated value of LVEF at time m, where time k occurs less than one week before time m. That is, model configuration service 332 may train machine learning model 354 based on paired sets of electrical cardiac training data 356 and LV dysfunction metric training data 358. The set of electrical cardiac data corresponding to each paired set of electrical cardiac training data and LV dysfunction metric training data may be collected from a patient less than one week before the set of LV dysfunction metric training data is collected from the patient. This means that once machine learning model 354 is trained, machine learning model 354 may accept a set of ECG data of electrical cardiac data 352 collected from a patient as an input output a confidence that a value of a metric of LV dysfunction of the patient will be less than a threshold value of the metric of LV dysfunction less than one week after the set of ECG data is collected from the patient.

In some examples, model configuration service 332 may train machine learning model 354 based on one or more sets of training data that each include a two or more sets of ECG data of electrical cardiac training data 356 collected from a patient paired with a set of LV dysfunction metric training data of LV dysfunction metric training data 358 collected from the patient. In some examples, each set of ECG data of the two or more sets of ECG data may correspond to a point in time or a window of time and the set of LV dysfunction metric training data may correspond to a point in time or a window of time. In some examples, the two or more sets of ECG data may each be collected from the patient before the set of LV dysfunction metric training data is collected from the patient. In some examples, the two or more sets of ECG data may each be collected from the patient after the set of LV dysfunction metric training data is collected from the patient. In some examples, some of the two or more sets of ECG data may be collected from the patient before the set of LV dysfunction metric training data is collected from the patient and some of the two or more sets of ECG data may be collected from the patient after the set of LV dysfunction metric training data is collected from the patient.

This means that once machine learning model 354 is trained, machine learning model 354 may output a value of a metric of LV dysfunction based on two or more sets of ECG data of electrical cardiac data 352 collected from a patient. In some examples, when machine learning model 354 is trained based on sets of training data that include more than one set of ECG data collected from the same patient, machine learning model 354 may be more robust as compared with when machine learning model 354 is trained based on sets of training data that include a single set of ECG data. The machine learning model 354 may, in some examples, operate according to the following equation:

$$F ( ECG ( {t}_{k} ) , ECG ( {t}_{k} - 1 ) , ECG ( {t}_{k} - 2 ) , ECG ( {t}_{k} + 1 ) ) ∼ EF ( tm ) \qquad ( eq . 2 )$$

$$where m - k , ( m - ( k - 1 ) ) , ( m - ( k - 2 ) ) , ( m - ( k + 1 ) ) < 1 week$$

As seen above in equation 2, machine learning model 354 (F) may accept as an input a set of ECG data of electrical cardiac data 352 collected from a patient at time k, a set of ECG data of electrical cardiac data 352 collected from the patient at time k−1, a set of ECG data of electrical cardiac data 352 collected from the patient at time k−2, and a set of ECG data of electrical cardiac data 352 collected from the patient at time k+1. Machine learning model 354 may output a value of a metric of LV dysfunction corresponding to the patient at time m. The value of the metric of LV dysfunction may, in some examples, represent a classification of whether the metric of LV dysfunction is less than a threshold metric of LV dysfunction. The value of the metric of LV dysfunction may, in some examples, represent a precise estimate of a value of the metric of LV dysfunction. In some examples, time k, time k−1, time k−2, and time k+1 may each be less than one week before time m.

Model configuration service 332 may, in some examples, use longitudinal data to train machine learning model 354 to output an ECG-based classification of whether LVEF is low or high (e.g., a confidence that LVEF is lower than an LVEF threshold). Model configuration service 332 may, in some examples, use longitudinal data to train machine learning model 354 to output an ECG-based determination of whether LVEF has significantly increased or significantly decreased. For example, machine learning model 354 may receive one or more sets of ECG data collected from a patient and output a determination of whether LVEF of the patient has increased more than an LVEF increase threshold or decreased more than an LVEF decrease threshold.

In some examples, data analyzer 330 may identify one or more parameters and/or characteristics indicated by electrical cardiac training data 356 such as heart rate, heart rate variability, arrhythmias, R-waves, T-waves, P-waves, or any combination thereof. Model configuration service 332 may develop machine learning model 354 to perform one or more LVEF classifications and/or one or more EF trend detections based on parameter ranges. For example, model configuration service 332 may train machine learning model 354 to process input electrical cardiac data differently based on a heart rate, a heart rate variability, or arrythmias.

In some examples, a patient's LVEF does not instantaneously change over a short period of time, and changes of LVEF might not occur frequently. This means that there may be hysteresis associated with the increasing and decreasing trends in LVEF. Data analyzer 330 may quantify two parameters from longitudinal EF data, hysteresis during an EF decrease, and hysteresis during an EF increase. These two parameters may be used as parameters for ECG-based EF change detection to detect physiologically realistic EF changes while decreasing a frequency of false positives.

In some examples, ICM ECG-LVEF paired measurements of training data 355 may be limited, but paired Holter ECG-LVEF measurements of training data 355 may be more readily available. In some examples, training data 355 may include one or more paired Holter/wearable ECG—LVEF datasets. In some examples, training data 355 may include one or more paired Holter/wearable ECG—ICM ECG datasets. That is, one or more sets of training data 355 may include ECG data collected from a patient via Holter monitor, a wearable device, an IMD (e.g., an ICM), or any combination thereof. For example, HMD 26 may mapMfrom wearable/Holter ECG data to an ICM ECG. In some examples, HMD 26 may map an ICM ECG to a Holter ECG according to the equation ICM ECG=M(Holter ECG). In some examples, HMD 26 may map an ICM ECG to a Holter ECG according to the equation ICM ECG=0.75*Lead II Holter ECG+0.25*Lead I Holter ECG. This mapping may be used on a dataset D1 to train machine learning model 354 to map M(D1 Holter ECG)˜=D1 EF. After machine learning model 354 is trained, machine learning model 354 may be used for subsequent ICM ECG-based EF classification.

Mortality of patients in heart failure may be correlated with contractile reserve. The term “contractile reserve” may refer to a difference between myocardial contractility and myocardial stress. In some examples, higher contractile reserve may be associated with lower mortality and lower contractile reserve may be associated with higher mortality. HMS 26 may estimate a contractile reserve for patients in heart failure based on ECG-derived LVEF, ECG-derived autonomic tone, medication information, activity information, or any combination thereof. In other words, HMS 26 may monitor a metric of LV dysfunction (e.g., LVEF) of patient 4 based on electrical cardiac data 352 (e.g., ECG data) to determine a contractile reserve for patient 4 to monitor a risk of mortality for patient 4.

ECG-derived LVEF may represent a metric for monitoring HF deterioration in patients with reduced LVEF. For example, when patient 4 has reduced LVEF, HMS 26 may track LVEF of patient 4 based on electrical cardiac data 352 collected from patient 4. HMS 26 may identify one or more trends in LVEF based on electrical cardiac data 352 collected from patient 4. When one or more trends in LVEF indicate decreasing LVEF, this may indicate heart failure deterioration. When one or more trends in LVEF indicate increasing LVEF, this may indicate heart failure improvement. In some examples, medical devices system 2 may collect electrical cardiac data using a multi-lead ICM system. To preserve battery longevity, an ICM may be used predominantly in a single-channel arrhythmia detection mode. For LVEF detection, a multi-channel ECG may be routinely recorded (e.g., once a day, once a weak, or according to any other interval) for LVEF classification via post-processing.

A system for ECG-based EF monitoring may include a single lead ICM device. During an implant procedure, a real-time LVEF estimation metric may be computed to determine if LVEF can be estimated based on data collected by the ICM in the implanted orientation. Since arrhythmia monitoring may work across multiple orientations of the ICM, the implant process can be optimized for ECG-based EF determination. In some examples, a system for ECG-based LVEF monitoring may, instead of analyzing routine ECG episodes from the device for LVEF estimation on a cloud (e.g., on computing system 20), the system may use a sensitive on-board algorithm (e.g., onboard IMD 10) to detect low LVEF or detect significant LVEF change. ECGs collected by IMD 10 may be post-processed by HMD 26 to reduce false positives.

HMS 26 may use ICM-ECG-based low LVEF detection to identify one or more patients who need further diagnostics and monitoring with echocardiogram and/or a cardiac MRI. A system for ECG-based LVEF may include a configurable device in terms of number or frequency of ECG measurements (e.g., on-demand or once every period of time), number of leads, and ECG sampling rate. This may allow the system to switch the EF monitoring mode per patient monitoring need (e.g., low-frequency monitoring before hospitalization for HF prediction, high frequency monitoring during hospitalization and 1-week post-discharge). HMS 26 may identify if HF patients have low or high LVEF for appropriate therapy follow up.

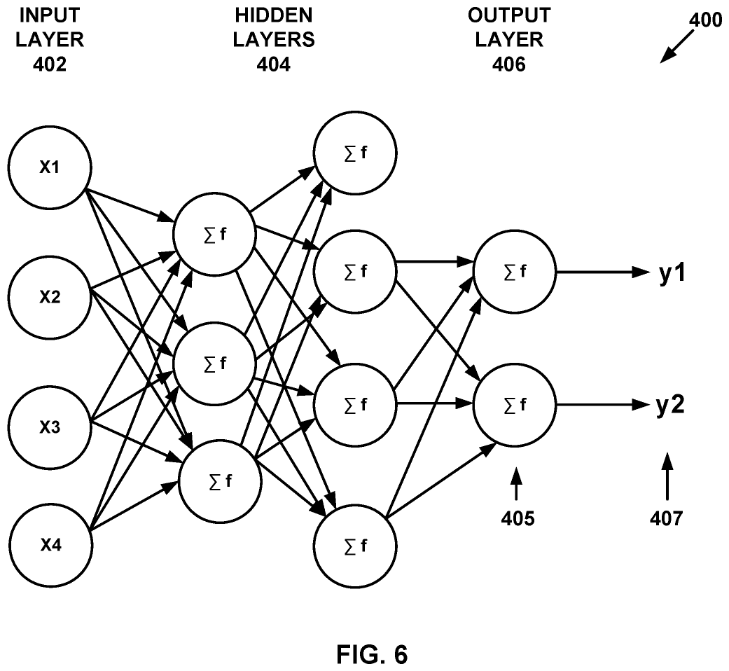

FIG. 6 is a conceptual diagram illustrating an example machine learning model 400 configured to output information corresponding to electrical cardiac data, in accordance with one or more techniques of this disclosure. Machine learning model 400 is an example of a deep learning model, or deep learning algorithm. One or more of IMD 10, computing devices 12, or computing system 20 (e.g., model configuration service 272 and/or model configuration service 332) may train, store, and/or utilize machine learning model 400, but other devices may apply inputs associated with a particular patient to machine learning model 400 in other examples. Some non-limiting examples of machine learning techniques include Bayesian probability models, Hawkes processes, Support Vector Machines, K-Nearest Neighbor algorithms, and Multi-layer Perceptron.

As shown in the example of FIG. 6, machine learning model 400 may include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may be parameters determined based on electrical cardiac data 184, 292, 352, including those described herein, and in some cases other parameter data 182, 290, 350.

Each of the input values for each node in the input layer 402 is provided to each node of hidden layer 404. In the example of FIG. 6, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between input physiological parameter values and one or more output values indicative of a health state of the patient. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be a value or values indicative a classification whether a metric of LV dysfunction is less than a threshold value and/or a determination of a precise value of a metric of LV dysfunction. By applying the data to a machine learning model, such as machine learning model 400, processing circuitry of medical device system 2 is able to determine the information corresponding to patient 4 with great specificity and sensitivity.

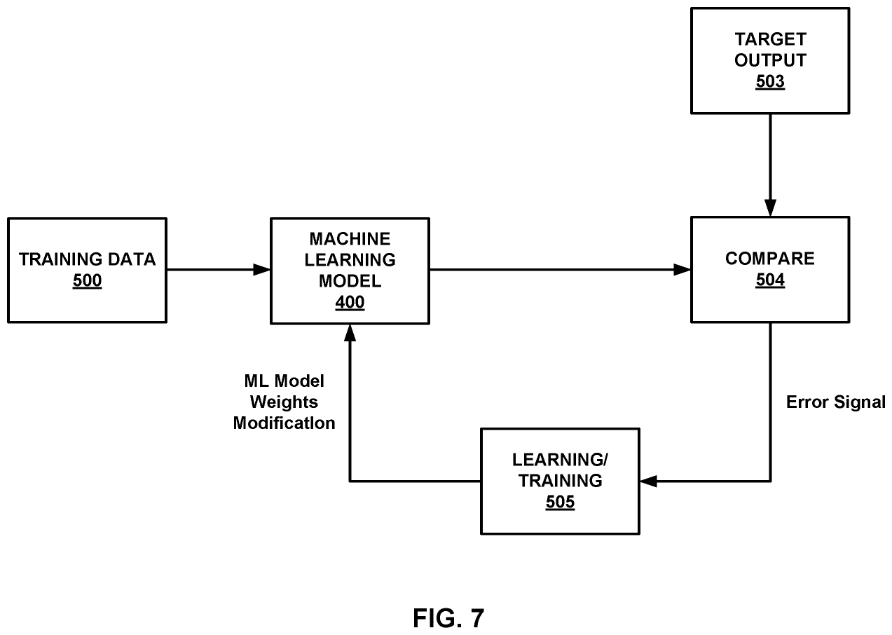

FIG. 7 is a block diagram illustrating an example of a machine learning model 400 being trained using supervised and/or reinforcement learning, in accordance with one or more techniques of this disclosure. Machine learning model 400 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry of one or more of IMD 10, external device 12, and/or computing system 20 initially trains the machine learning model 400 based on training data 500. Training data may, in some examples, include training data 355 of FIG. 5. An output of the machine learning model 400 may be compared 504 to the target output 503, e.g., as determined based on the label. Based on an error signal representing the comparison, the processing circuitry implementing a learning/training function 505 may send or apply a modification to weights of machine learning model 400 or otherwise modify/update the machine learning model 400. For example, one or more of IMD 10, external device 12, and/or computing system 20 may, for each training instance in the training set 500, modify machine learning model 400 to change an output generated by the machine learning model 400 in response to data applied to the machine learning model 400.

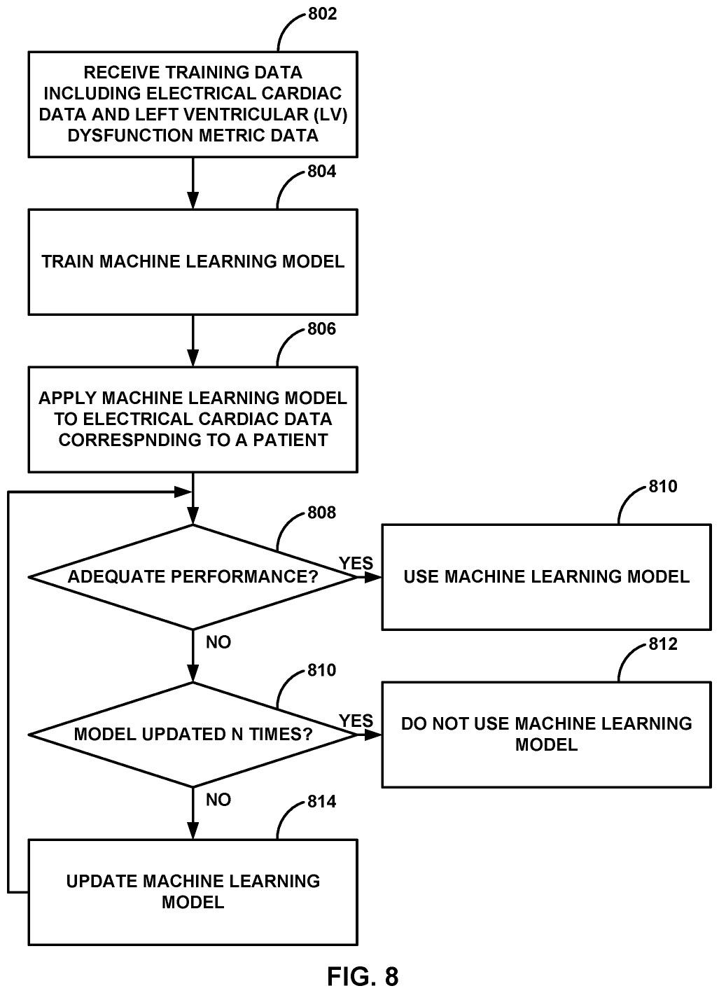

FIG. 8 is a flow diagram illustrating an example method for determining whether to use a machine learning model to process electrical cardiac data collected from a patient, in accordance with one or more techniques of this disclosure. FIG. 8 is described with respect to medical device system 2 of FIG. 1. However, the techniques of FIG. 8 may be performed by different components of medical device system 2 or by additional or alternative medical device systems.

HMS 26 may train a model and personalize and/or calibrate the model to specific patients. For example, HMS 26 may train a machine learning model to implement an ECG-based algorithm (e.g., for low LVEF classification) and validate the machine learning model with a large dataset. HMS 26 may apply the machine learning model to electrical cardiac data collected from a patient for prospective use. In some examples, HMS 26 may obtain the patient's ECG and paired EF. When the machine learning model performs adequately with respect to the patient, the machine learning model may be is used as it currently stands. When the machine learning model does not perform adequately with respect to a patient, the machine learning model may be tuned with the ECG-LVEF paired data measurement and up to N more ECG-LVEF paired data measurements from that patient. When the algorithm parameters can be tuned for that patient with the patient-specific data to achieve adequate performance, the personalized algorithm may be used for the patient. If the machine learning model is updated over N iterations and remains insufficient, the machine learning model may be flagged as not applicable to the patient, and the data may be collated for an overall model update. “Adequate performance” may correspond to an ability to detect low LVEF or an ability to detect significant changes in LVEF.

HMS 26 may receive training data including electrical cardiac data and LV dysfunction metric data (802). In some examples, the electrical cardiac data and the LV dysfunction metric data may correspond to a patient, such as patient 4. HMS 26 may train a machine learning model (804) based on the electrical cardiac data and LV dysfunction metric data corresponding to patient 4. In some examples, HMS 26 may train the machine learning model based on a set of training data including one or more paired sets of electrical cardiac data and LV dysfunction metric data corresponding to one or more human patients other than patient 4. HMS 26 may, in some examples, receive a set of incoming electrical cardiac data from patient 4.

HMS 26 may apply, to the incoming electrical cardiac data corresponding to patient 4, the machine learning model (806) to determine a value of a metric of LV dysfunction. In some examples, the machine learning model may represent a classification model configured to output a confidence that the value of a metric of LV dysfunction is lower than a threshold value of the metric of LV dysfunction. In some examples, the machine learning model may represent a regression model configured to output an estimate of a precise value of a metric of LV dysfunction.

HMS 26 may determine, based on the output from the machine learning model, whether the machine learning model performs adequately with respect to patient 4 (808). In some examples, to determine whether the machine learning model performs adequately with respect to patient 4, HMS 26 may compare the output from the machine learning model with LV dysfunction metric data corresponding to patient 4. When the machine learning model performs adequately with respect to patient 4 (“YES” at block 808), HMS 26 may use the machine learning model to process electrical cardiac data corresponding to patient 4.

When the machine learning model does not perform adequately with respect to patient 4 (“NO” at block 808), HMS 26 may determine whether the machine learning model has been updated at least N times (810). When the machine learning model has been updated at least Ntimes (“YES” at block 810), HMS 26 may decline to use the machine learning model to process data corresponding to patient 4 (812). When the machine learning model has not been updated at least Ntimes (“NO” at block 810), HMS 26 may update the machine learning model (814). In some examples, to update the machine learning model, HMS 26 may re-train the machine learning model with additional training data corresponding to patient 4. In some examples, to update the machine learning model, HMS 26 may re-train the machine learning model with additional training data corresponding to one or more patients other than patient 4. When HMS 26 updates the machine learning model, the process of FIG. 8 may return to block 808.

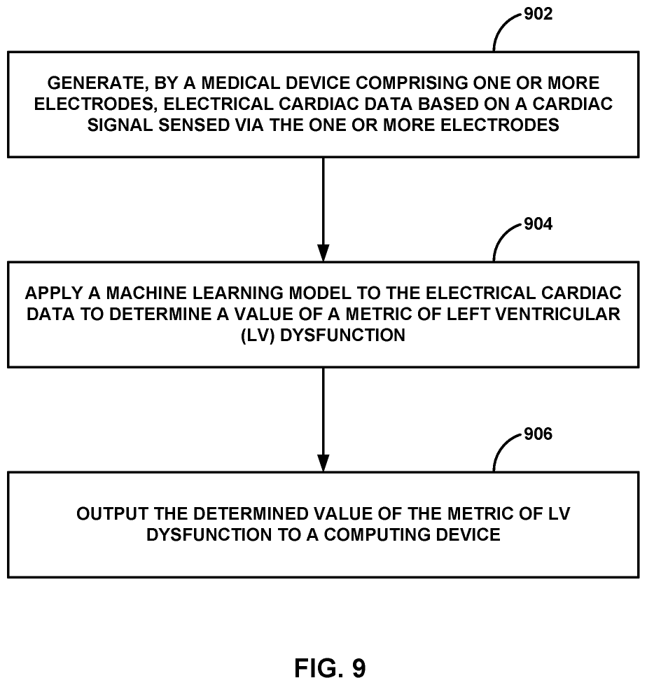

FIG. 9 is a flow diagram illustrating an example method for applying a machine learning model to determine a value of a metric of LV dysfunction, in accordance with one or more techniques of this disclosure. FIG. 9 is described with respect to medical device system 2 of FIG. 1. However, the techniques of FIG. 9 may be performed by different components of medical device system 2 or by additional or alternative medical device systems.

IMD 10 may generate electrical cardiac data based on a cardiac signal sensed by IMD 10 via one or more electrodes (902). HMS 26 may apply a machine learning model to the electrical cardiac data to determine a value of a metric of LV dysfunction (904). The techniques of this disclosure are not limited to HMS 26 applying a machine learning model to electrical cardiac data. Computing devices 12A, 12B and/or IMD 10 may apply the machine learning model in some cases. In some examples, the machine learning model is trained based on a plurality of sets of training data. Each set of training data of the plurality of sets of training data may include a set of training electrical cardiac data and information indicating one or more values of the metric of LV dysfunction corresponding to the set of training electrical cardiac data. HMS 26 may output a determined value of the metric of LV dysfunction to a computing device (e.g., computing devices 12A, 12B) (906).

The following numbered clauses may demonstrate one or more aspects of the disclosure.

Clause 1: A medical device system includes a medical device system comprising a medical device comprising one or more electrodes and configured to generate electrical cardiac data based on a cardiac signal sensed from a patient via the one or more electrodes, a memory configured to store a machine learning model and a plurality of sets of training data, and processing circuitry in communication with the memory. The processing circuitry is configured to apply the machine learning model to the electrical cardiac data to determine a value of a metric of left ventricular (LV) dysfunction. The machine learning model is trained based on the plurality of sets of training data, wherein each set of training data of the plurality of sets of training data includes a set of training electrical cardiac data and information indicating one or more values of the metric of LV dysfunction corresponding to the set of training electrical cardiac data. Additionally, the processing circuitry is configured to output the determined value of the metric of LV dysfunction to a computing device.

Clause 2: The medical device system of clause 1, wherein the electrical cardiac data comprises a plurality of sets of electrical cardiac data, wherein to apply the machine learning model to the electrical cardiac data to determine the value of the metric of LV dysfunction, the processing circuitry is configured to: apply the machine learning model to a set of electrical cardiac data of the plurality of sets of electrical cardiac data to determine the value of the metric of LV dysfunction which corresponds to the set of electrical cardiac data of the plurality of sets of electrical cardiac data, and wherein the processing circuitry is further configured to: apply the machine learning model to each other set of electrical cardiac data of the plurality of sets of electrical cardiac data to determine a value of the metric of LV dysfunction corresponding to each other set of electrical cardiac data of the plurality of sets of electrical cardiac data; and output the determined value of the metric of LV dysfunction corresponding to each other set of electrical cardiac data of the plurality of sets of electrical cardiac data to the computing device.

Clause 3: The medical device system of any of clauses 1-2, wherein the medical device further comprises an accelerometer, wherein the medical device is further configured to generate motion data based on a motion signal sensed by the accelerometer, and wherein the processing circuitry is further configured to: determine, based on the motion data, a motion value indicating an activity level of the patient, wherein the motion value corresponds to the determined value of the metric of LV dysfunction; and determine whether to output an alert based on the motion value and the determined value of the metric of LV dysfunction.

Clause 4: The medical device system of clause 3, wherein to determine whether to output the alert, the processing circuitry is configured to: determine whether the value of the metric of LV dysfunction is lower than a threshold metric of LV dysfunction; determine whether the motion value is greater than a threshold motion value; and determine whether to output the alert based on whether the value of the metric of LV dysfunction is lower than the threshold metric of LV dysfunction and whether the motion value is greater than the threshold motion value.

Clause 5: The medical device system of any of clauses 1-4, wherein the processing circuitry is further configured to train the machine learning model based on the plurality of sets of training data, and wherein by training the machine learning model based on the plurality of sets of training data, the processing circuitry is configured to cause the machine learning model to recognize one or more patterns corresponding to the metric of LV dysfunction and one or more characteristics of electrical cardiac data.

Clause 6: The medical device system of any of clauses 1-5, wherein the processing circuitry is further configured to label each set of training data of the plurality of sets of training data.

Clause 7: The medical device system of clause 6, wherein to label each set of training data of the plurality of sets of training data, the processing circuitry is configured to: identify, for each set of training data of the plurality of sets of training data, one or more characteristics of the set of training electrical cardiac data of the set of training data; and label the set of training electrical cardiac data of each set of training data of the plurality of sets of training data with the one or more characteristics of the set of training electrical cardiac data.

Clause 8: The medical device system of clause 7, wherein the one or more characteristics of the set of training electrical cardiac data include any one or more of: one or more R-waves, one or more P-waves, one or more T-waves, a heart rate corresponding to the set of training electrical cardiac data, a heart rate variability corresponding to set of training electrical cardiac data, and an arrythmia indicated by the set of training electrical cardiac data.

Clause 9: The medical device system of any of clauses 6-7, wherein to label each set of training data of the plurality of sets of training data, the processing circuitry is configured to: identify, in the information indicating one or more values of the metric of LV dysfunction of the set of training data, a time corresponding to each value of the one or more values of the metric of LV dysfunction; and associate the time corresponding to each value of the one or more values of the metric of LV dysfunction with a time of the set of training electrical cardiac data.

Clause 10: The medical device system of any of clauses 1-9, wherein to apply the machine learning model to the electrical cardiac data to determine the value of LV dysfunction, the processing circuitry is configured to apply the machine learning model to the electrical cardiac data to determine a confidence that the value of LV dysfunction is lower than a threshold value of LV dysfunction.

Clause 11: The medical device system of any of clauses 1-10, wherein the metric of LV dysfunction comprises ejection fraction.

Clause 12: A method of operating a medical device system comprising a medical device comprising one or more electrodes and configured to generate electrical cardiac data based on a cardiac signal sensed from a patient via the one or more electrodes, the method comprising: applying, by processing circuitry of the medical device system, a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data; and outputting, by the processing circuitry, the determined value of the metric of LV dysfunction to a computing device.

Clause 13: The method of clause 12, wherein the medical device further comprises an accelerometer, wherein the medical device is further configured to generate motion data based on a motion signal sensed by the accelerometer, and wherein the method further comprises: determining, by the processing circuitry based on the motion data, a motion value indicating an activity level of the patient, wherein the motion value corresponds to the determined value of the metric of LV dysfunction; and determining, by the processing circuitry, whether to output an alert based on the motion value and the determined value of the metric of LV dysfunction.

Clause 14: The method of any of clauses 12-13, wherein the medical device further comprises an accelerometer, wherein the medical device is further configured to generate motion data based on a motion signal sensed by the accelerometer, and wherein the method further comprises: determining, by the processing circuitry based on the motion data, a motion value indicating an activity level of the patient, wherein the motion value corresponds to the determined value of the metric of LV dysfunction; and determining, by the processing circuitry, whether to output an alert based on the motion value and the determined value of the metric of LV dysfunction.

Clause 15: The method of clause 14, wherein determining whether to output the alert comprises: determining, by the processing circuitry, whether the value of the metric of LV dysfunction is lower than a threshold metric of LV dysfunction; determining, by the processing circuitry, whether the motion value is greater than a threshold motion value; and determining, by the processing circuitry, whether to output the alert based on whether the value of the metric of LV dysfunction is lower than the threshold metric of LV dysfunction and whether the motion value is greater than the threshold motion value.

Clause 16: The method of any of clauses 12-15, wherein the method further comprises training, by the processing circuitry, the machine learning model based on the plurality of sets of training data, and wherein by training the machine learning model based on the plurality of sets of training data, the method comprises causing, by the processing circuitry, the machine learning model to recognize one or more patterns corresponding to the metric of LV dysfunction and one or more characteristics of electrical cardiac data.

Clause 17: The method of any of clauses 12-16, wherein the method further comprises labeling, by the processing circuitry, each set of training data of the plurality of sets of training data.

Clause 18: The method of clause 17, wherein labeling each set of training data of the plurality of sets of training data comprises: identifying, by the processing circuitry for each set of training data of the plurality of sets of training data, one or more characteristics of the set of training electrical cardiac data of the set of training data; and labeling, by the processing circuitry, the set of training electrical cardiac data of each set of training data of the plurality of sets of training data with the one or more characteristics of the set of training electrical cardiac data.

Clause 19: The method of any of clauses 17-18, wherein labeling each set of training data of the plurality of sets of training data comprises: identifying, by the processing circuitry in the information indicating one or more values of the metric of LV dysfunction of the set of training data, a time corresponding to each value of the one or more values of the metric of LV dysfunction; and associating, by the processing circuitry, the time corresponding to each value of the one or more values of the metric of LV dysfunction with a time of the set of training electrical cardiac data.

Clause 20: A non-transitory computer-readable storage medium includes program instructions that, when executed by processing circuitry of a medical device system comprising one or more electrodes and configured to generate electrical cardiac data based on a cardiac signal sensed from a patient via the one or more electrodes, cause the processing circuitry to: apply a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data; and output the determined value of the metric of LV dysfunction to a computing device.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A medical device system comprising:
a medical device comprising one or more electrodes and configured to generate electrical cardiac data based on a cardiac signal sensed from a patient via the one or more electrodes;
a memory configured to store a machine learning model and a plurality of sets of training data; and
processing circuitry in communication with the memory, wherein the processing circuitry is configured to:
apply the machine learning model to the electrical cardiac data to determine a value of a metric of left ventricular (LV) dysfunction, wherein the machine learning model is trained based on the plurality of sets of training data, wherein each set of training data of the plurality of sets of training data includes a set of training electrical cardiac data and information indicating one or more values of the metric of LV dysfunction corresponding to the set of training electrical cardiac data; and
output the determined value of the metric of LV dysfunction to a computing device.

2. The medical device system of claim 1, wherein the electrical cardiac data comprises a plurality of sets of electrical cardiac data, wherein to apply the machine learning model to the electrical cardiac data to determine the value of the metric of LV dysfunction, the processing circuitry is configured to:
apply the machine learning model to a set of electrical cardiac data of the plurality of sets of electrical cardiac data to determine the value of the metric of LV dysfunction which corresponds to the set of electrical cardiac data of the plurality of sets of electrical cardiac data, and
wherein the processing circuitry is further configured to:
apply the machine learning model to each other set of electrical cardiac data of the plurality of sets of electrical cardiac data to determine a value of the metric of LV dysfunction corresponding to each other set of electrical cardiac data of the plurality of sets of electrical cardiac data; and
output the determined value of the metric of LV dysfunction corresponding to each other set of electrical cardiac data of the plurality of sets of electrical cardiac data to the computing device.

3. The medical device system of claim 1, wherein the medical device further comprises an accelerometer, wherein the medical device is further configured to generate motion data based on a motion signal sensed by the accelerometer, and wherein the processing circuitry is further configured to:
determine, based on the motion data, a motion value indicating an activity level of the patient, wherein the motion value corresponds to the determined value of the metric of LV dysfunction; and
determine whether to output an alert based on the motion value and the determined value of the metric of LV dysfunction.

4. The medical device system of claim 3, wherein to determine whether to output the alert, the processing circuitry is configured to:
determine whether the value of the metric of LV dysfunction is lower than a threshold metric of LV dysfunction;
determine whether the motion value is greater than a threshold motion value; and
determine whether to output the alert based on whether the value of the metric of LV dysfunction is lower than the threshold metric of LV dysfunction and whether the motion value is greater than the threshold motion value.

5. The medical device system of claim 1,
wherein the processing circuitry is further configured to train the machine learning model based on the plurality of sets of training data, and
wherein by training the machine learning model based on the plurality of sets of training data, the processing circuitry is configured to cause the machine learning model to recognize one or more patterns corresponding to the metric of LV dysfunction and one or more characteristics of electrical cardiac data.

6. The medical device system of claim 1, wherein the processing circuitry is further configured to label each set of training data of the plurality of sets of training data.

7. The medical device system of claim 6, wherein to label each set of training data of the plurality of sets of training data, the processing circuitry is configured to:
identify, for each set of training data of the plurality of sets of training data, one or more characteristics of the set of training electrical cardiac data of the set of training data; and
label the set of training electrical cardiac data of each set of training data of the plurality of sets of training data with the one or more characteristics of the set of training electrical cardiac data.

8. The medical device system of claim 7, wherein the one or more characteristics of the set of training electrical cardiac data include any one or more of: one or more R-waves, one or more P-waves, one or more T-waves, a heart rate corresponding to the set of training electrical cardiac data, a heart rate variability corresponding to set of training electrical cardiac data, and an arrythmia indicated by the set of training electrical cardiac data.

9. The medical device system of claim 6, wherein to label each set of training data of the plurality of sets of training data, the processing circuitry is configured to:
identify, in the information indicating one or more values of the metric of LV dysfunction of the set of training data, a time corresponding to each value of the one or more values of the metric of LV dysfunction; and
associate the time corresponding to each value of the one or more values of the metric of LV dysfunction with a time of the set of training electrical cardiac data.

10. The medical device system of claim 1, wherein to apply the machine learning model to the electrical cardiac data to determine the value of LV dysfunction, the processing circuitry is configured to apply the machine learning model to the electrical cardiac data to determine a confidence that the value of LV dysfunction is lower than a threshold value of LV dysfunction.

11. The medical device system of claim 1, wherein the metric of LV dysfunction comprises ejection fraction.

12. A method of operating a medical device system comprising a medical device comprising one or more electrodes and configured to generate electrical cardiac data based on a cardiac signal sensed from a patient via the one or more electrodes, the method comprising:
applying, by processing circuitry of the medical device system, a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data; and
outputting, by the processing circuitry, the determined value of the metric of LV dysfunction to a computing device.

13. The method of claim 12, wherein the medical device further comprises an accelerometer, wherein the medical device is further configured to generate motion data based on a motion signal sensed by the accelerometer, and wherein the method further comprises:
determining, by the processing circuitry based on the motion data, a motion value indicating an activity level of the patient, wherein the motion value corresponds to the determined value of the metric of LV dysfunction; and
determining, by the processing circuitry, whether to output an alert based on the motion value and the determined value of the metric of LV dysfunction.

14. The method of claim 12, wherein the medical device further comprises an accelerometer, wherein the medical device is further configured to generate motion data based on a motion signal sensed by the accelerometer, and wherein the method further comprises:
determining, by the processing circuitry based on the motion data, a motion value indicating an activity level of the patient, wherein the motion value corresponds to the determined value of the metric of LV dysfunction; and
determining, by the processing circuitry, whether to output an alert based on the motion value and the determined value of the metric of LV dysfunction.

15. The method of claim 14, wherein determining whether to output the alert comprises:
determining, by the processing circuitry, whether the value of the metric of LV dysfunction is lower than a threshold metric of LV dysfunction;
determining, by the processing circuitry, whether the motion value is greater than a threshold motion value; and
determining, by the processing circuitry, whether to output the alert based on whether the value of the metric of LV dysfunction is lower than the threshold metric of LV dysfunction and whether the motion value is greater than the threshold motion value.

16. The method of claim 12,
wherein the method further comprises training, by the processing circuitry, the machine learning model based on the plurality of sets of training data, and
wherein by training the machine learning model based on the plurality of sets of training data, the method comprises causing, by the processing circuitry, the machine learning model to recognize one or more patterns corresponding to the metric of LV dysfunction and one or more characteristics of electrical cardiac data.

17. The method of claim 12, wherein the method further comprises labeling, by the processing circuitry, each set of training data of the plurality of sets of training data.

18. The method of claim 17, wherein labeling each set of training data of the plurality of sets of training data comprises:
identifying, by the processing circuitry for each set of training data of the plurality of sets of training data, one or more characteristics of the set of training electrical cardiac data of the set of training data; and
labeling, by the processing circuitry, the set of training electrical cardiac data of each set of training data of the plurality of sets of training data with the one or more characteristics of the set of training electrical cardiac data.

19. The method of claim 17, wherein labeling each set of training data of the plurality of sets of training data comprises:
identifying, by the processing circuitry in the information indicating one or more values of the metric of LV dysfunction of the set of training data, a time corresponding to each value of the one or more values of the metric of LV dysfunction; and
associating, by the processing circuitry, the time corresponding to each value of the one or more values of the metric of LV dysfunction with a time of the set of training electrical cardiac data.

20. A non-transitory computer-readable storage medium comprising program instructions that, when executed by processing circuitry of a medical device system comprising one or more electrodes and configured to generate electrical cardiac data based on a cardiac signal sensed from a patient via the one or more electrodes, cause the processing circuitry to:
apply a machine learning model to the electrical cardiac data to determine a value of a metric of left ventricular (LV) dysfunction, wherein the machine learning model is trained based on a plurality of sets of training data, wherein each set of training data of the plurality of sets of training data includes a set of training electrical cardiac data and information indicating one or more values of the metric of LV dysfunction corresponding to the set of training electrical cardiac data, wherein the processing circuitry is in communication with a memory of the medical device system, the memory configured to store the machine learning model and the plurality of sets of training data; and
output the determined value of the metric of LV dysfunction to a computing device.
